(19) United States
(12) Patent Application Publication
(10) Pub. No.: US 2012/0009183 A1
(43) Pub. Date: Jan. 12, 2012

(54) METHOD FOR TREATMENT OF PANCREATITIS

(75) Inventors: Louis Anthony Tartaglia, Newton, MA (US); Thomas Michael Barnes, Brookline, MA (US); Robert Mark Coopersmith, Chestnut Hill, MA (US); Scott Edward Malstrom, Reading, MA (US); David William White, Plymouth County, MA (US); Luz-Maria Guzman, Watertown, MA (US)

(73) Assignee: Ore Pharmaceuticals Inc., Gaithersburg, MD (US)

(21) Appl. No.: 13/171,058

(22) Filed: Jun. 28, 2011

Related U.S. Application Data
(63) Continuation of application No. 12/248,259, filed on Oct. 9, 2008, now abandoned.
(60) Provisional application No. 60/978,814, filed on Oct. 10, 2007.

(51) Int. Cl. A61K 39/395; A61P 29/00; A61P 1/18; A61K 31/4172

(52) U.S. Cl. 424/133.1; 514/400

(57) ABSTRACT

A method for treating acute or chronic pancreatitis in a subject comprises administering to the subject a therapeutically effective amount of an ACE2 inhibitor.

This application claims the benefit of U.S. application Ser. No. 12/248,259, filed Oct. 9, 2008, which claims the benefit of U.S. provisional application Ser. No. 60/978,814 filed on Oct. 10, 2007, the entire disclosure of which is incorporated herein by reference.

### FIELD OF THE INVENTION

The present invention relates to pharmacotherapy for acute or chronic pancreatitis.

### BACKGROUND

### General Background of Pancreatitis

The pancreas is a large gland located behind the stomach and adjacent to the duodenum (the first section of the small intestine). Its primary functions are (a) exocrine, viz. secretion of digestive enzymes into the small intestine to aid in digestion of carbohydrates, proteins and fats; and (b) endocrine, viz. production and release of hormones including insulin and glucagon into the bloodstream, where they are involved in regulation of blood glucose metabolism.

The term “pancreatitis” covers a variety of diseases in which the pancreas becomes inflamed. This is believed to occur when powerful digestive enzymes, in particular trypsinogen, are activated before secretion into the duodenum, and attack the pancreatic tissues. Pancreatitis includes acute and chronic forms that differ in pathology but can have similar symptoms.

Acute pancreatitis is a sudden, usually painful inflammation that occurs over a short period of time, and can be precipitated by a variety of causal factors including gallstones, heavy alcohol use, medications, infections, metabolic disorders, trauma and surgery, or can occur without known cause. Acute pancreatitis is a rather common complication following endoscopic retrograde cholangiopancreatography (ERCP). Severity of acute pancreatitis can range from mild abdominal discomfort to severe illness that can lead to serious, sometimes life-threatening complications. In most but by no means all cases, treatment with analgesics for management of pain is sufficient. Treatment can also include removal of causal factors (e.g., control of infection with antibiotics, surgery to remove gallstones, abstinence from alcohol, etc.) as appropriate. Over 80% of patients recover completely after receiving the appropriate treatment.

Chronic pancreatitis (CP) occurs most commonly after an episode of acute pancreatitis and involves ongoing or recurrent inflammation of the pancreas, often leading to extensive scarring or fibrosis. CP causes progressive and irreversible damage to the pancreas and surrounding tissues. Calcification of pancreatic tissues is common and often diagnostic of CP. In over 70% of cases, CP is associated with excessive and prolonged alcohol consumption. While alcoholism is the most common cause of CP, other causes include metabolic disorders and, more rarely, genetic disposition (hereditary pancreatitis). In some cases, no cause can be determined.

Damage to the pancreas from excessive alcohol use can remain asymptomatic for many years, but eventually loss of pancreatic function, resulting in digestion and blood sugar abnormalities, usually accompanied by pain, develops. Reduced secretion of digestive enzymes causes malabsorption of nutrients from food, leading often to abnormal feces and weight loss. Damage to insulin-producing cells in the pancreas can result in diabetes. Pain due to CP is typically experienced as a constant pain in the upper abdomen that radiates to the back, and can be severe enough to be disabling. Pain can be accompanied by nausea and vomiting. As the disease progresses, attacks of pain last longer and become more frequent.

CP increases risk of pancreatic cancer, the most deadly of all gastrointestinal system malignancies, by 10- to 20-fold (see Farrow et al. (2004) Ann. Surg. 239:763-771).

Treatment options for CP are presently limited, and in view of the irreversible nature of the damage caused by CP the best that can often be achieved is stabilization of the condition. Especially where CP can be traced to alcohol abuse, complete abstinence from alcohol is required. In patients having alcoholism, this may require psychiatric therapy and/or participation in support groups. Dietary changes (e.g., reduced fat) can be implemented to reduce demand for the depleted enzyme supply from the damaged pancreas. Enzyme supplements can be administered to aid digestion. Concomitantly, pain can be managed with analgesics, although conventional analgesics are often inadequate and present risks of addiction or other adverse effects when administered chronically.

Information on CP, its symptoms, pathology and treatment can be found in various print and internet sources, including for example a review article by Steer et al. (1995) NEJM 332(22): 1482-1490.

### BACKGROUND OF THE INVENTION

Angiotensin II (Ang II), a member of the renin-angiotensin system (RAS) and the primary product of angiotensin converting enzyme (ACE), is known to exert pro-inflammatory effects in a variety of tissues, via its type 1 and type 2 receptors (AT₁ and AT₂ respectively) and, in many cases, ultimately through activation of nuclear factor κB (NF-κB).

In the classical pathway of Ang II synthesis in the circulating RAS, the precursor of Ang II is angiotensinogen, which is principally produced in the liver and then cleaved by renin to form angiotensin I (Ang I), which is converted by ACE into Ang II that is carried to various target cells via the circulatory system. See, e.g., Inokuchi et al. (2005) Gut 54:349-356, and sources cited therein. In addition, tissue-specific renin-angiotensin systems have been identified in many organs, suggesting that various tissues have the ability to synthesize Ang II independently of circulating RAS, including kidney, brain, aorta, adrenal gland, heart, stomach and colon.

Donoghue et al. (2000) Circ. Res. 87:1-9 reported identification of a carboxypeptidase related to ACE from sequencing of a human heart failure ventricle cDNA library. This carboxypeptidase, ACE2, was stated to be the first known human homolog of ACE. The authors further reported that the metalloprotease catalytic domains of ACE2 and ACE are 42% identical, and that, in contrast to the more ubiquitous ACE, ACE2 transcripts are found only in heart, kidney, and testis in the 23 human tissues examined.

U.S. Pat. No. 6,194,556 to Acton et al. discloses novel genes encoding ACE2. Therapeutics, diagnostics and screening assays based on these genes are also disclosed.

Harmer et al. (2002) FEBS Lett. 532:107-110 reported quantitative mapping of the transcriptional expression profile of ACE2 (and the two isoforms of ACE) in 72 human tissues. The study reportedly confirmed that ACE2 expression is high in renal and cardiovascular tissues. It was further reported that ACE2 shows comparably high levels of expression in the gastrointestinal system, in particular in ileum, duodenum, jejunum, cecum and colon. The authors proposed that in probing functional significance of ACE2, some consideration should be given to a role in gastrointestinal physiology and pathophysiology.

Rice et al. (2003) Bull. Br. Soc. Cardiovasc. Res. 16(2):5-11 reviewed potential functional roles of ACE2 and indicated that its expression is mainly localized in testis, kidney, heart and intestines.

Ferreira & Santos (2005) Braz. J. Med. Biol. Res. 38:499-507 have summarized important pathways of the RAS, including roles of ACE and ACE2, as shown in FIG. 1 herein.

As evidence of implication of Ang II, the main product of ACE, in a variety of pro-inflammatory effects, see for example:
Phillips & Kagiyama (2002) Curr. Opin. Investig. Drugs 3(4):569-577, who reviewed literature showing Ang II to be a key factor, via NF-κB activation, in promoting inflammation, inter alia, in atherosclerosis;
Costanzo et al. (2003) J. Cell Physiol. 195(3):402-410, who reported up-regulation by Ang II of endothelial cell adhesion molecules involved in atherosclerosis, via inflammatory cytokines through NF-κB activation;
Sanz-Rosa et al. (2005) Am. J. Physiol. Heart Circ. Physiol. 288:H111-H115, who reported that blocking the AT₁ receptor reduces the level of vascular and circulating inflammatory mediators such as NF-κB and TNF-α in spontaneous hypertension;
Esteban et al. (2004) J. Am. Soc. Nephrol. 15:1514-1529, who reported that Ang II, via AT₁ and AT₂, activates NF-κB and thereby promotes inflammation in obstructed kidney; and
Inokuchi et al. (2005), supra, who reported that in angiotensinogen gene knockout mice, which have low levels of Ang II, inflammatory colitis induced by 2,4,6-trinitrobenzenesulfonic acid (TNBS) is ameliorated, and that blocking the AT₁ receptor also ameliorated TNBS-induced colitis.

Evidence for pro-inflammatory effects of Ang II specifically in pancreatitis is found, for example, in the publications individually cited below.

Nagashio et al. (2004) Am. J. Physiol. Gastrointest. Liver Physiol. 287:170-177 reported that deleting the AT₁ receptor in mice reduces pancreatic stellate cell (PSC) activation and fibrosis in acute pancreatitis induced by cerulein. In this regard, it is noted that the RAS is present in pancreas and is enhanced in acute pancreatitis, that repeated episodes of acute pancreatitis can lead to increasing destruction of exocrine parenchyma tissues of the pancreas and replacement by fibrotic tissue, that such increasing damage eventually results in chronic pancreatitis (CP), and that PSCs play a central role in fibrogenesis in the pancreas.

Tsang et al. (2004) Regul. Pept. 119(3):213-219 reported that the selective AT₁ receptor antagonist losartan (but not a specific AT₂ antagonist) markedly reduced severity of acute pancreatitis induced by cerulein.

Kuno et al. (2003) Gastroenterology 124:1010-1019 reported that the ACE inhibitor lisinopril attenuated pancreatic fibrosis in an animal model of CP (male WBN/Kob rats, that spontaneously develop CP). Lisinopril was reported to suppress expression of TGFβ₁ mRNA, resulting in prevention of PSC activation.

Yamada et al. (2003) J. Pharmacol. Exp. Ther. 307:17-23 reported that the AT₁ receptor antagonist candesartan alleviated CP and fibrosis in the WBN/Kob rat model of CP, by suppressing overexpression of TGFβ₁, resulting in prevention of PSC activation.

Yamada et al. (2005) J. Pharmacol. Exp. Ther. 313:36-45 reported that combination therapy with the ACE inhibitor lisinopril and the AT₁ receptor antagonist candesartan at low doses synergistically suppressed inflammation and fibrosis in the WBN/Kob rat model of CP.

The proinflammatory effects of the ACE product Ang II have been found to be generally counterbalanced by ACE2 in various studies involving ACE2 disruption and/or mutants lacking the ACE2 gene. See for example:
Crackower et al. (2002) Nature 417(6891):822-828, who reported that disruption of ACE2 or deletion of the ACE2 gene in various rat models raises the level of Ang II;
Huentelman et al. (2005) Exp. Physiol. 90(5):783-790, who reported that injection of a vector encoding ACE2 protects wild-type mice against Ang II induced cardiac hypertrophy and fibrosis; and
Imai et al. (2005) Nature 436(7047):112-116, who reported that deletion of the ACE gene or giving ACE2 protein to wild-type mice protects against acid-induced acute lung injury.

The primary product of ACE2, namely angiotensin (1-7), via its receptor (Mas), has generally been found to oppose functions of the ACE product Ang II. See for example:
Guy et al. (2005) Biochim. Biophys. Acta 1751(1):2-8, who reviewed literature indicating inter alia that ACE2 regulates heart and kidney function by control of Ang II levels relative to angiotensin (1-7), and may therefore counterbalance the effects of ACE within the RAS;
Ferreira & Santos (2005), supra, who reviewed literature indicating inter alia that ACE inhibitor benefits may be partly mediated by the ACE2 product angiotensin (1-7), plasma levels of which are greatly increased following chronic administration of ACE inhibitors;
Mendes et al. (2005) Regul. Pept. 125(1-3):29-34, who reported that infusion of angiotensin (1-7) reduces Ang II levels in the heart and postulated that such reduction may contribute to beneficial effects of angiotensin (1-7); and
Tallant & Clark (2003) Hypertension 42:574-579, who reported that angiotensin (1-7) reduces smooth muscle growth after vascular injury, and counteracts stimulation by Ang II of growth and mitogen activated protein (MAP) kinase activity in rat aortic vascular smooth muscle cells.

Thus ACE2 activity appears to counterbalance inflammatory effects of Ang II in a variety of tissues, whether by increasing angiotensin (1-7) levels or reducing Ang II levels or both.

In one scenario, therefore, promotion of ACE2 activity might be of interest for reducing inflammation in gastrointestinal diseases such as acute and chronic pancreatitis. Huentelman et al. (2004) 44:903-906 proposed, similarly, that in vivo activation of ACE2 could lead to protection and successful treatment for hypertension and other cardiovascular diseases, by counterbalancing the potent vasoconstrictive effects of Ang II.

Agents that inhibit rather than promote ACE2 activity have been described in the art. For example, Huentelman et al. (2004), supra, reported efforts to identify ACE2 inhibitory compounds that inhibit infection by SARS-CoV, the coronavirus responsible for severe acute respiratory syndrome (SARS), for which ACE2 has been found to be a functional receptor. Among the compounds so identified was NAAE (N-(2-aminoethyl)-1-aziridine-ethanamine).

U.S. Pat. No. 6,900,033 to Parry et al. discloses peptides comprising specific amino acid sequences that are said to specifically bind to ACE2 protein or ACE2-like polypeptides. It is proposed at column 53, lines 63-65 thereof that “an abnormally high a[n]giotensin II level could result from abnormally low activity of ACE-2” and at column 63, lines 21-32 thereof that “ACE-2 binding polypeptides . . . which activate ACE-2-induced signal transduction can be administered to an animal to treat, prevent or ameliorate a disease or disorder associated with aberrant ACE-2 expression, lack of ACE-2 function, aberrant ACE-2 substrate expression, or lack of ACE-2 substrate function. These ACE-2 binding polypeptides may potentiate or activate either all or a subset of the biological activities of ACE-2-mediated substrate action . . . ”. At column 74, lines 28-37 thereof, it is stated: “Further, the invention [whether by activation or inhibition of ACE2 not specified] can be useful to treat, prevent, or ameliorate diseases and disorders . . . of the pancreas including, but not limited to, acute pancreatitis, chronic pancreatitis (acute necrotizing pancreatitis, alcoholic pancreatitis), neoplasms (adenocarcinoma of the pancreas, cystadenocarcinoma, insulinoma, gastrinoma, and glucagonoma, cystic neoplasms, islet-cell tumors, pancreoblastoma), and other pancreatic diseases (e.g., cystic fibrosis, cyst (pancreatic pseudocyst, pancreatic fistula, insufficiency)).” Separately, ACE2 binding peptides that are reported to inhibit ACE2 in vitro are identified in Table 2 at columns 127-130 thereof.

Huang et al. (2003) J. Biol. Chem. 278(18):15532-15540 reported that one such ACE2 inhibitory peptide, namely DX600, exhibited an ACE2 Kᵢ value of 2.8 nM.

Li et al. (2005) Am. J. Physiol. Renal Physiol. 288:F353-F362 reported that DX600 blocked Ang I mediated generation of angiotensin (1-7) in rat nephron segments.

U.S. Pat. No. 6,632,830 to Acton et al. discloses compounds comprising a zinc coordinating moiety and an amino acid mimicking moiety, said to be useful for modulating activity of ACE2. More particularly, there are disclosed ACE2 inhibiting compounds of a generic formula presented therein. Such compounds are said to be useful for treating an “ACE-2 associated state” in a patient. “ACE-2 associated states” are said to include high blood pressure and diseases and disorders related thereto, in particular arterial hypertension, congestive heart failure, chronic heart failure, left ventricular hypertrophy, acute heart failure, myocardial infarction and cardiomyopathy; states associated with regulating smooth cell proliferation, in particular smooth muscle cell proliferation; kidney diseases and disorders; other hyperadrenergic states; kinetensin associated conditions including those caused by, or contributed to by, abnormal histamine release, for example in local or systemic allergic reactions including eczema, asthma and anaphylactic shock; infertility or other disorders relating to gamete maturation; cognitive disorders; disorders associated with bradykinin and des-Arg bradykinin; and “other examples” (column 36, lines 58-67 thereof) that are said to include “SIRS . . . , sepsis, polytrauma, inflammatory bowel disease, acute and chronic pain, bone destruction in rheumatoid and osteo arthritis and periodontal disease, dysmenorrhea, premature labor, brain edema following focal injury, diffuse axonal injury, stroke, reperfusion injury and cerebral vasospasm after subarachnoid hemorrhage, allergic disorders including asthma, adult respiratory distress syndrome, wound healing and scar formation.”

Dales et al. (2002) J. Am. Chem. Soc. 124:11852-11853 reported ACE2 IC₅₀ values of a range of such compounds. The most active of these was compound 16, identified therein as having the formula

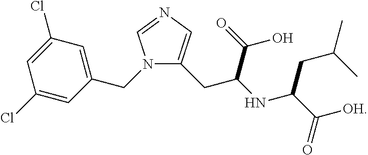

All four stereoisomers of compound 16 were prepared, and the greatest potency was reported for the S,S-isomer, which reportedly had an IC₅₀ for ACE2 of 0.44 nM. The S,S-isomer of the above compound, 2-[1-carboxy-2-[3-(3,5-dichlorobenzyl)-3H-imidazol-4-yl]-ethylamino]-4-methylpentanoic acid, also known as MLN-4760, is referred to herein as GL1001.

A need continues to exist for new pharmacotherapies for pancreatitis, to replace or supplement the limited range of treatment options now available to the prescribing physician and the pancreatitis patient. The need is especially great for CP, but exists also for acute pancreatitis, particularly in patients who are not responsive to analgesic therapy, for whom surgery is not an acceptable option, or who suffer recurrent acute pancreatitis, which frequently leads to CP. To our knowledge, a compound with ACE2 inhibition properties such as GL1001 has not heretofore been contemplated for treatment of pancreatitis or studied in an animal model of pancreatitis.

### SUMMARY OF THE INVENTION

It has now surprisingly been found that expression of ACE2 mRNA in pancreas tissue is dramatically upregulated in a chronic pancreatitis (CP) disease condition. High levels of ACE2 mRNA expression thus appearing to be a factor associated with CP, it is contemplated that reduction of ACE2 activity in the pancreas, for example by administration of an ACE2 inhibitor, could provide therapeutic benefit to a CP patient. The effect of an ACE2 inhibitor on the renin-angiotensin system (RAS) might be predicted to involve increase in level of Ang II (see FIG. 1), which as indicated above is implicated in a variety of pro-inflammatory effects. Contrary to such prediction, it has now further surprisingly been found that activation of NF-κB, a key mediator for synthesis of pro-inflammatory cytokines, is not promoted but inhibited by an ACE2 inhibitor; and furthermore that this inhibition of NF-κB activation is accompanied by decreased levels of pro-inflammatory cytokines and increased levels of anti-inflammatory cytokines in plasma, indicating potential for systemic control of chronic inflammatory diseases such as CP. It is contemplated that the benefits of an ACE2 inhibitor in CP are applicable to pancreatitis generally, including acute pancreatitis.

Accordingly, there is now provided a method for treating pancreatitis, especially CP, in a subject, comprising administering to the subject a therapeutically effective amount of an ACE2 inhibitor.

There is further provided a method for reducing risk of pancreatic cancer in a subject having CP, comprising administering an ACE2 inhibitor to the subject in an amount effective at least to slow progression of the CP.

There is still further provided a method for managing pancreatitis, especially CP, in a subject, comprising administering a therapeutically effective amount of an ACE2 inhibitor adjunctively with at least one additional agent for managing pancreatitis or a condition associated therewith.

There is still further provided a method for treating alcohol-related pancreatitis, especially alcohol-related CP, comprising administering a therapeutically effective amount of an ACE2 inhibitor to the subject in conjunction with abstinence from alcohol consumption by the subject.

Other embodiments, including particular aspects of the embodiments summarized above, will be evident from the detailed description that follows.

### BRIEF DESCRIPTION OF THE DRAWINGS

FIG. 1 is a schematic representation of enzymatic pathways of the renin-angiotensin system (RAS) involved in generation of angiotensin peptides. Key:

ACE=angiotensin converting enzyme;

AMP=aminopeptidase;

Ang=angiotensin;

AT₁=angiotensin II type 1 receptor;

AT₁₋₇=angiotensin (1-7) receptor;

AT₂=angiotensin II type 2 receptor;

D-Amp=dipeptidyl aminopeptidase;

IRAP=insulin regulated aminopeptidase;

NEP=neutral endopeptidase 24.11;

PCP=prolyl carboxypeptidase;

PEP=prolyl endopeptidase.

(From Ferreira & Santos (2005), supra.)

FIG. 2 is a graphical representation of inhibition by GL1001 of TNFα-induced activation of NF-κB in recombinant HeLa reporter cells, as described in Example 2.

FIG. 3 is a graphical representation of inhibition by GL1001 of in vivo basal NF-κB dependent transcription in recombinant reporter mice, as described in Example 3.

FIG. 4 is a graphical representation of inhibition by GL1001 of in vivo LPS induced NF-κB signaling in mice, as described in Example 4. Mice were pretreated with GL1001 (subcutaneous) for 1 hour before LPS treatment. All mice treated with 0.1 mg/kg LPS (i.v.). Abdominal ROI used for quantitative data (2.76×3.7 cm). Mean±SEM, n=5 for each group; * p<0.05, ** p<0.01, ANOVA and student t-test between treatments and controls.

FIG. 5 is a graphical representation of inhibition by GL1001 of in vivo LPS induced NF-κB signaling in mice, as described in Example 4. Male mice were pretreated with GL1001 and LPS, and were imaged, as in FIG. 4. Mean±SEM, n=5 for each group; * p<0.05, ** p<0.01, ANOVA and student t-test between treatments and controls.

FIG. 6 is a graphical representation of inhibition by GL1001 of LPS induced NF-κB dependent transcription in selected organs of recombinant reporter mice, as described in Example 4.

FIG. 7 is a graphical representation of cytokine levels in plasma of LPS-treated mice receiving GL1001 at two doses, or vehicle control, based on results of the study described in Example 6. Mean±SEM; ANOVA was used to test for significance between the vehicle control and GL1001 treatment groups, * p<0.05, ** p<0.01, *** p<0.001.

FIG. 8 is a graphical representation of results of in vivo imaging and abdominal ROI analysis. Mice were imaged using biophotonic imaging as discussed in Example 5. A region of interest encompassing the abdominal cavity was used for photon analysis. Mean±SEM, n=4 control water group, n=10 for DSS+saline and DSS+100 mg/kg GL1001. ANOVA and student t-test were used to test for significance between the DSS+saline and DSS+GL1001 treatment groups.

FIG. 9 is a graphical representation of results showing fluid intake per mouse. Water bottles were weighed daily and fluid consumption was represented as grams of water consumed per mouse, as described in Example 5. Mean±SEM, n=4 control water group, n=10 for DSS+saline and DSS+100 mg/kg GL1001. ANOVA and student t-test were used to test for significance between the DSS+saline and DSS+GL1001 treatment groups; * p<0.05.

FIG. 10 is a graphical representation of results showing inhibition of IBD (as measured by IBD activity index) by GL1001. The index was determined for each mouse at each time point as described in Table 3 of Example 5. Mean±SEM; ANOVA and student t-test were used to test for significance between the DSS+saline control and DSS+GL1001 treatment groups, * p<0.05, ** p<0.01.

FIG. 11 is a graphical representation of results showing body weight change in response to DSS treatment, as described in Example 5. Calculated weight changes are shown over the time course of the experiment. Mean±SEM; ANOVA and student t-test were used to test for significance between the DSS+saline control and DSS+GL1001 treatment groups, * p<0.05, ** p<0.01.

FIG. 12 is a graphical representation of organ/body weight measurements indicating that GL1001 treated mice have a reduction in DSS induced organ weight increase as compared to DSS controls, as described in Example 5. Mean±SEM; ANOVA and student t-test were used to test for significance between the DSS+saline control and DSS+GL1001 treatment groups, ** p<0.01.

FIG. 13 is a graphical representation of results showing that cecum and large intestine demonstrate increased luciferase in the DSS control treatment group in a luciferase organ lysate analysis. Assay performed as described in Example 5. Mean±SEM; prox=proximal, dist=distal.

### DETAILED DESCRIPTION

Studies have provided evidence of efficacy of compounds or antibodies other than ACE2 inhibitors in animal models of acute pancreatitis and CP. See, for example, the review articles individually cited below and incorporated by reference herein without admission that they constitute prior art to the present invention.
Bang et al. (2008) World J. Gastroenterol. 14(19):2968-2976.
Nagashio & Otsuki (2007) J. Pancreas 8(4 Suppl.):495-500.
Talukdar & Tandon (2007) J. Gastroenterol. Hepatol. 23:34-41.

Such studies have shown, for example, that in acute pancreatitis models, treatment with the antioxidant N-acetylcysteine; and in CP models, treatment with the peroxisome proliferation-activated receptor γ (PPARγ) ligand troglitazone, the serine protease inhibitor camostat mesylate, the ACE inhibitor lisinopril, the AT₁ receptor antagonist candesartan, the cyclooxygenase-2 (COX-2) inhibitor rofecoxib, or soluble TGFβ receptor (to block TGFβ signaling) have a beneficial effect.

Various therapeutic methods are described herein, all involving administration of an ACE2 inhibitor to a subject having pancreatitis. The invention is described herein with particular reference to CP; however, in most aspects it is contemplated that benefits are obtainable not only in CP but also in acute pancreatitis.

Any ACE2 inhibitor can be used. In general it will be found useful to select an ACE2 inhibitor having relatively high affinity for ACE2, as expressed for example by IC₅₀ or Kᵢ, whether measured in vitro or in vivo. In one embodiment, the ACE2 inhibitor selected is one that exhibits in vitro an ACE2 IC₅₀ and/or an ACE2 Kᵢ not greater than about 1000 nM, for example not greater than about 500 nM, not greater than about 250 nM, or not greater than about 100 nM.

ACE2 inhibitors are known to differ not only in their affinity for ACE2 but also in their selectivity for binding to ACE2 as opposed to the more ubiquitous ACE. In one embodiment, the ACE2 inhibitor exhibits selectivity for ACE2 versus ACE, as expressed by the ratio of IC₅₀(ACE) to IC₅₀(ACE2), of at least about 10², for example at least about 10³, or at least about 10⁴.

Large-molecule (“biological”) and small-molecule ACE2 inhibitors can be used. Large-molecule ACE2 inhibitors can be, for example, peptides, antibodies or interfering RNA (iRNA) based molecules. Examples of peptide ACE2 inhibitors, and methods for preparing them, can be found for example in above-cited U.S. Pat. No. 6,900,033, which is incorporated herein by reference in its entirety. Peptide compounds exhibiting relatively strong inhibition of ACE2 illustratively include those having peptide sequences identified as DX-512, DX-513, DX-524, DX-525, DX-529, DX-531, DX-599, DX-600, DX-601 and DX-602 in U.S. Pat. No. 6,900,033.

For many purposes it will be found preferable to use a non-peptide or “small molecule” ACE2 inhibitor. The term “small-molecule” herein means having a molecular weight not greater than about 1000, for example not greater than about 800 or not greater than about 600, excluding any conjugate (e.g., a peptide conjugate present to modify absorption or tissue distribution properties of the ACE2 inhibitor) or, in the case of salt forms, counterions. Such compounds tend to be easier to prepare, especially on a large or commercial scale, have lower cost, and present fewer problems in administration and delivery to the active site in the body. In various embodiments, therefore, the ACE2 inhibitor comprises a small-molecule compound or a pharmaceutically acceptable salt thereof or a prodrug thereof.

Illustratively, an ACE2 inhibitor can be of a non-peptide type disclosed generically in above-cited U.S. Pat. No. 6,632,830, which is incorporated herein by reference in its entirety, including any of the specific compounds disclosed therein along with methods of preparation thereof. In one embodiment, the compound comprises a zinc coordinating moiety and an amino acid mimicking moiety.

More specifically, the compound can have the formula

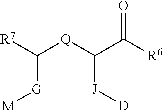

as disclosed in U.S. Pat. No. 6,632,830, wherein
R⁶ is hydroxyl or a protecting prodrug moiety;
R⁷ is hydrogen, carboxylic acid, ether, alkoxy, an amide, a protecting prodrug moiety, hydroxyl, thiol, heterocyclyl, alkyl or amine;
Q is CH₂, O, NH or NR³, wherein R³ is substituted or unsubstituted C₁₋₅ branched or straight chain alkyl, C₂₋₅ branched or straight chain alkenyl, substituted or unsubstituted acyl, aryl or a C₃₋₈ ring;
G is a covalent bond or a CH₂, ether, thioether, amine or carbonyl linking moiety;
M is heteroaryl, substituted with at least one subanchor moiety comprising a substituted or unsubstituted cycloalkyl or aryl ring, linked thereto through a sublinking moiety (CH₂)ₙ or (CH₂)ₙO(CH₂)ₙ where n is an integer from 0 to 3;
J is a bond or a substituted or unsubstituted alkyl, alkenyl or alkynyl moiety; and
D is alkyl, alkenyl, alkynyl, aryl or heteroaryl, optionally linked to G or M to form a ring.

In one embodiment, in the above formula for the compound, R⁶ is hydroxyl, R⁷ is carboxylic acid, Q is NH and G is CH₂.

In one embodiment, in the above formula for the compound, the heteroaryl group of M is imidazolyl, thienyl, triazolyl, pyrazolyl or thiazolyl. Independently of the selection of heteroaryl group, the subanchor moiety according to this embodiment is C₃₋₆ cycloalkyl, phenyl, methylenedioxyphenyl, naphthalenyl, or phenyl having 1 to 3 substituents independently selected from halo, C₁₋₆ alkyl, C₃₋₆ cycloalkyl, trifluoromethyl, C₁₋₆ alkoxy, trifluoromethoxy, phenyl, cyano, nitro and carboxylic acid groups, and is linked to the heteroaryl group through a (CH₂)ₙ or (CH₂)O(CH₂) sublinking moiety, where n is an integer from 0 to 3.

In one embodiment, in the above formula for the compound, J is a bond or CH₂ moiety and D is C₁₋₆ alkyl, C₃₋₆ cycloalkyl or phenyl.

In a more particular embodiment, in the formula for the compound:
R⁶ is hydroxyl;
R⁷ is carboxylic acid;
Q is NH;
G is CH₂;
M is imidazolyl, thienyl, triazolyl, pyrazolyl or thiazolyl, linked through a (CH₂)ₙ or (CH₂)O(CH₂) sublinking moiety, where n is an integer from 0 to 3, to a subanchor moiety that is C₃₋₆ cycloalkyl, phenyl, methylenedioxyphenyl, naphthalenyl, or phenyl having 1 to 3 substituents independently selected from halo, C₁₋₆ alkyl, C₃₋₆ cycloalkyl, trifluoromethyl, C₁₋₆ alkoxy, trifluoromethoxy, phenyl, cyano, nitro and carboxylic acid groups;
J is a bond or CH₂ moiety; and
D is C₁₋₆ alkyl, C₃₋₆ cycloalkyl or phenyl.

According to any of the above embodiments the compound can be present in any enantiomeric configuration, e.g., (R,R), (R,S), (S,R) or (S,S), or as a mixture, for example a racemic mixture, of enantiomers. However, in general it is found preferable that the compound be present in the (S,S)-configuration. In one embodiment, the compound is in the (S,S)-configuration and is substantially enantiomerically pure. For example, the compound can exhibit an enantiomeric purity of at least about 90%, at least about 95%, at least about 98% or at least about 99%, by weight of all enantiomeric forms of the compound present.

Illustrative compounds specifically disclosed in U.S. Pat. No. 6,632,830 include the following, each of which can be in any enantiomeric form, illustratively in the (S,S)-configuration:
2-[1-carboxy-2-[3-(4-trifluoromethylbenzyl)-3H-imidazol-4-yl]ethylamino]-4-methylpentanoic acid;
2-[1-carboxy-2-[3-naphthalen-1-ylmethyl-3H-imidazol-4-yl]ethylamino]-4-methylpentanoic acid;
2-[1-carboxy-2-[3-(4-chlorobenzyl)-3H-imidazol-4-yl]ethylamino]-4-methylpentanoic acid;
2-[1-carboxy-2-[3-(3,4-dichlorobenzyl)-3H-imidazol-4-yl]ethylamino]-4-methylpentanoic acid;
2-[1-carboxy-2-[3-(4-cyanobenzyl)-3H-imidazol-4-yl]ethylamino]-4-methylpentanoic acid;
2-[1-carboxy-2-[3-(3-chlorobenzyl)-3H-imidazol-4-yl]ethylamino]-4-methylpentanoic acid;
2-[1-carboxy-2-[3-(3,5-dichlorobenzyl)-3H-imidazol-4-yl]ethylamino]-4-methylpentanoic acid;
2-[1-carboxy-2-[3-(4-methylbenzyl)-3H-imidazol-4-yl]ethylamino]-4-methylpentanoic acid;
2-[1-carboxy-2-[3-(3,4-dimethylbenzyl)-3H-imidazol-4-yl]ethylamino]-4-methylpentanoic acid;
2-[1-carboxy-2-[3-(3-methylbenzyl)-3H-imidazol-4-yl]ethylamino]-4-methylpentanoic acid;
2-[1-carboxy-2-[3-(3,5-dimethylbenzyl)-3H-imidazol-4-yl]ethylamino]-4-methylpentanoic acid;
2-[1-carboxy-2-[3-(4-trifluoromethoxybenzyl)-3H-imidazol-4-yl]ethylamino]-4-methylpentanoic acid;
2-[1-carboxy-2-[3-(4-isopropylbenzyl)-3H-imidazol-4-yl]ethylamino]-4-methylpentanoic acid;
2-[1-carboxy-2-[3-(4-tert-butylbenzyl)-3H-imidazol-4-yl]ethylamino]-4-methylpentanoic acid;
2-[1-carboxy-2-[3-(4-nitrobenzyl)-3H-imidazol-4-yl]ethylamino]-4-methylpentanoic acid;
2-[1-carboxy-2-[3-(2,3-dimethoxybenzyl)-3H-imidazol-4-yl]ethylamino]-4-methylpentanoic acid;
2-[1-carboxy-2-[3-(2,3-difluorobenzyl)-3H-imidazol-4-yl]ethylamino]-4-methylpentanoic acid;
2-[1-carboxy-2-[3-(2,3-dichlorobenzyl)-3H-imidazol-4-yl]ethylamino]-4-methylpentanoic acid;
2-[1-carboxy-2-[3-(3-trifluoromethylbenzyl)-3H-imidazol-4-yl]ethylamino]-4-methylpentanoic acid;
2-[2-(3-benzo[1,3]dioxol-5-ylmethyl-3H-imidazol-4-yl)-1-carboxyethylamino]-4-methylpentanoic acid;
2-[1-carboxy-2-[3-(2-cyclohexylethyl)-3H-imidazol-4-yl]ethylamino]-4-methylpentanoic acid;
2-[1-carboxy-2-[3-phenethyl-3H-imidazol-4-yl]ethylamino]-4-methylpentanoic acid;
2-[1-carboxy-2-[3-(3-iodobenzyl)-3H-imidazol-4-yl]ethylamino]-4-methylpentanoic acid;
2-[1-carboxy-2-[3-(3-fluorobenzyl)-3H-imidazol-4-yl]ethylamino]-4-methylpentanoic acid;
2-[1-carboxy-2-[3-benzyloxymethyl-3H-imidazol-4-yl]ethylamino]-4-methylpentanoic acid;
2-[1-carboxy-2-[3-(4-butylbenzyl)-3H-imidazol-4-yl]ethylamino]-4-methylpentanoic acid;
2-[1-carboxy-2-[3-(2-methylbenzyl)-3H-imidazol-4-yl]ethylamino]-4-methylpentanoic acid;
2-[1-carboxy-2-[2-phenylthiazol-4-yl]ethylamino]-4-methylpentanoic acid;
2-[1-carboxy-2-[1-benzyl)-1H-pyrazol-4-yl]ethylamino]-4-methylpentanoic acid; and
2-[1-carboxy-2-[3-(2-methylbiphenyl-3-ylmethyl)-3H-imidazol-4-yl]ethylamino]-4-methylpentanoic acid.

As in all embodiments, any of the above compounds can be present in the above form or in the form of a pharmaceutically acceptable salt thereof, or a prodrug thereof.

The present invention is illustrated herein by particular reference to (S,S)-2-[1-carboxy-2-[3-(3,5-dichlorobenzyl)-3H-imidazol-4-yl]-ethylamino]-4-methylpentanoic acid, otherwise known as GL1001, which is the (S,S)-enantiomer of a compound having the formula

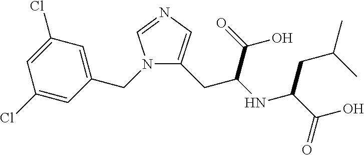

as disclosed for example by Dales et al. (2002), supra, together with a process for preparing such a compound. In brief, this process comprises treating (S)-histidine methyl ester with Boc₂O to provide a fully protected histidine derivative. The N-3 imidazole nitrogen is then selectively alkylated using the triflate of 3,5-dichlorobenzyl alcohol. Following Boc deprotection, reductive amination between the resulting alkylated histidine derivative and a β-ketoester furnishes a diester amine compound, which by hydrolysis yields 2-[1-carboxy-2-[3-(3,5-dichlorobenzyl)-3H-imidazol-4-yl]-ethylamino]-4-methylpentanoic acid as a mixture of diastereomers. The diastereomers can be separated and purified using HPLC and crystallization.

Other processes can be used to prepare GL1001, including without limitation processes described in above-referenced U.S. Pat. No. 6,632,830.

Methods provided herein are useful in treating chronic pancreatitis arising from a variety of identifiable causes as well as idiopathic chronic pancreatitis. For example, the method can be used in a subject having chronic pancreatitis induced by exposure to or ingestion of toxins, including alcohol-induced pancreatitis; hereditary chronic pancreatitis; chronic pancreatitis as a development from recurrent acute pancreatitis in a subject having a history thereof; or tropical pancreatitis.

A “subject” herein is a warm-blooded animal, generally a mammal such as, for example, a cat, dog, horse, cow, pig, mouse, rat or primate, including a human. In one embodiment the subject is a human, for example a patient having clinically diagnosed chronic pancreatitis. Animal models in experimental investigations relevant to human disease are also examples of “subjects” herein, and can include for example rodents (e.g., mouse, rat, guinea pig), lagomorphs (e.g., rabbit), carnivores (e.g., cat, dog), or nonhuman primates (e.g., monkey, chimpanzee). Further, the subject can be an animal (for example a domestic, farm, working, sporting or zoo animal) in veterinary care.

Certain compounds useful according to the present invention have acid and/or base moieties that, under suitable conditions, can form salts with suitable acids. For example, GL1001 has two acid moieties that, under suitable conditions, can form salts with suitable bases, and an amino group that, under suitable conditions, can form salts with suitable acids. Internal salts can also be formed. The compound can be used in its free acid/base form or in the form of an internal salt, an acid addition salt or a salt with a base.

Acid addition salts can illustratively be formed with inorganic acids such as mineral acids, for example sulfuric acid, phosphoric acids or hydrohalic (e.g., hydrochloric or hydrobromic) acids; with organic carboxylic acids such as (a) C₁₋₄ alkanecarboxylic acids which may be unsubstituted or substituted (e.g., halosubstituted), for example acetic acid, (b) saturated or unsaturated dicarboxylic acids, for example oxalic, malonic, succinic, maleic, fumaric, phthalic or terephthalic acids, (c) hydroxycarboxylic acids, for example ascorbic, glycolic, lactic, malic, tartaric or citric acids, (d) amino acids, for example aspartic or glutamic acids, or (e) benzoic acid; or with organic sulfonic acids such as C₁₋₄ alkanesulfonic acids or arylsulfonic acids which may be unsubstituted (e.g., halosubstituted), for example methanesulfonic acid or p-toluenesulfonic acid.

Salts with bases include metal salts such as alkali metal or alkaline earth metal salts, for example sodium, potassium or magnesium salts; or salts with ammonia or an organic amine such as morpholine, thiomorpholine, piperidine, pyrrolidine, a mono-, di- or tri-lower alkyl amine, for example ethylamine, tert-butylamine, diethylamine, diisopropylamine, triethylamine, tributylamine or dimethylpropylamine, or a mono-, di- or tri-(hydroxy lower alkyl)amine, for example monoethanolamine, diethanolamine or triethanolamine.

Alternatively, a prodrug of the compound or a salt of such prodrug can be used. A prodrug is a compound, typically itself having weak or no pharmaceutical activity, that is cleaved, metabolized or otherwise converted in the body of a subject to an active compound, in this case an ACE2 inhibitory compound. Examples of prodrugs are esters, particularly alkanoyl esters and more particularly C₁₋₆ alkanoyl esters. Other examples include carbamates, carbonates, ketals, acetals, phosphates, phosphonates, sulfates and sulfonates.

The ACE2 inhibitor should be administered in a therapeutically effective amount. What constitutes a therapeutically effective amount depends on a number of factors, including the particular subject's age and body weight, the nature, stage and severity of the disease, the particular effect sought (e.g., slowing or reversal of progress of the disease or a pathological process such as fibrosis associated therewith, alleviation of symptoms, etc.) and other factors, but for most subjects a dosage amount of about 0.5 to about 5000 mg/day, more typically about 5 to about 1000 mg/day, will be found suitable. In particular embodiments, the dosage employed is about 10 to about 800 mg/day, about 50 to about 750 mg/day or about 100 to about 600 mg/day; illustratively about 50, about 100, about 150, about 200, about 250, about 300, about 350, about 400, about 450, about 500, about 550, about 600, about 650, about 700 or about 750 mg/day.

Where a salt or prodrug of the ACE2 inhibitory compound is used, the amount administered should be an amount delivering a daily dosage of the compound as set forth above.

The above dosages are given on a per diem basis but should not be interpreted as necessarily being administered on a once daily frequency. Indeed the compound, or salt or prodrug thereof, can be administered at any suitable frequency, for example as determined conventionally by a physician taking into account a number of factors, but typically about four times a day, three times a day, twice a day, once a day, every second day, twice a week, once a week, twice a month or once a month. The compound, or salt or prodrug thereof, can alternatively be administered more or less continuously, for example by parenteral infusion in a hospital setting. In some situations a single dose may be administered, but more typically administration is according to a regimen involving repeated dosage over a treatment period. In such a regimen the daily dosage and/or frequency of administration can, if desired, be varied over the course of the treatment period, for example introducing the subject to the compound at a relatively low dose and then increasing the dose in one or more steps until a full dose is reached.

The treatment period is generally as long as is needed to achieve a desired outcome. In some situations it will be found useful to administer the drug intermittently, for example for treatment periods of days, weeks or months separated by non-treatment periods. Such intermittent administration can be timed, for example, to correspond to exacerbations of the disease.

Administration can be by any suitable route, including without limitation oral, buccal, sublingual, intranasal, intraocular, rectal, vaginal, transdermal or parenteral (e.g., intradermal, subcutaneous, intramuscular, intravenous, intra-arterial, intratracheal, intraventricular, intraperitoneal, etc.) routes, and including by inhalation or implantation.

While it can be possible to administer the compound, or a salt or prodrug thereof unformulated as active pharmaceutical ingredient (API) alone, it will generally be found preferable to administer the API in a pharmaceutical composition that comprises the API and at least one pharmaceutically acceptable excipient. The excipient(s) collectively provide a vehicle or carrier for the API. Pharmaceutical compositions adapted for all possible routes of administration are well known in the art and can be prepared according to principles and procedures set forth in standard texts and handbooks such as those individually cited below.
USIP, ed. (2005) Remington: The Science and Practice of Pharmacy, 21st ed., Lippincott, Williams & Wilkins.
Allen et al. (2004) Ansel's Pharmaceutical Dosage Forms and Drug Delivery Systems, 8th ed., Lippincott, Williams & Wilkins.

Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present invention include, without limitation, solutions, suspensions, powders, granules, tablets, capsules, pills, lozenges, chews, creams, ointments, gels, liposome preparations, nanoparticulate preparations, injectable preparations, enemas, suppositories, inhalable powders, sprayable liquids, aerosols, patches, depots and implants.

Illustratively, in a liquid formulation suitable, for example, for parenteral, intranasal or oral delivery, the API can be present in solution or suspension, or in some other form of dispersion, in a liquid medium that comprises a diluent such as water. Additional excipients that can be present in such a formulation include a tonicifying agent, a buffer (e.g., a tris, phosphate, imidazole or bicarbonate buffer), a dispersing or suspending agent and/or a preservative. Such a formulation can contain micro- or nanoparticulates, micelles and/or liposomes. A parenteral formulation can be prepared in dry reconstitutable form, requiring addition of a liquid carrier such as water or saline prior to administration by injection.

For rectal delivery, the API can be present in dispersed form in a suitable liquid (e.g., as an enema), semi-solid (e.g., as a cream or ointment) or solid (e.g., as a suppository) medium. The medium can be hydrophilic or lipophilic.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. The diluent or diluents selected preferably exhibit suitable flow properties and, where tablets are desired, compressibility.

Lactose, microcrystalline cellulose and starch, either individually or in combination, are particularly useful diluents.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC or hypromellose), hydroxypropylcellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

Croscarmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used. Tablets can be uncoated or can comprise a core that is coated, for example with a nonfunctional film or a release-modifying or enteric coating. Capsules can have hard or soft shells comprising, for example, gelatin and/or HPMC, optionally together with one or more plasticizers.

A pharmaceutical composition useful herein typically contains the compound or salt or prodrug thereof in an amount of about 1% to about 99%, more typically about 5% to about 90% or about 10% to about 60%, by weight of the composition. A unit dosage form such as a tablet or capsule can conveniently contain an amount of the compound providing a single dose, although where the dose required is large it may be necessary or desirable to administer a plurality of dosage forms as a single dose. Illustratively, a unit dosage form can comprise the compound in an amount of about 10 to about 800 mg, for example about 50 to about 750 mg or about 100 to about 600 mg; or, in particular illustrative instances, about 50, about 100, about 150, about 200, about 250, about 300, about 350, about 400, about 450, about 500, about 550, about 600, about 650, about 700 or about 750 mg.

Unless the context demands otherwise, the term “treat,” “treating” or “treatment” herein includes preventive or prophylactic use of an agent, for example an ACE2 inhibitor, in a subject at risk of, or having a prognosis including, acute or chronic pancreatitis, as well as use of such an agent in a subject already experiencing acute or chronic pancreatitis, as a therapy to alleviate, relieve, reduce intensity of or eliminate one or more symptoms of the disease or an underlying cause thereof. Thus treatment includes (a) preventing a condition or disease from occurring in a subject that may be predisposed to the condition or disease but in whom the condition or disease has not yet been diagnosed; (b) inhibiting the condition or disease, including retarding or arresting its development; and/or (c) relieving, alleviating or ameliorating the condition or disease, or primary or secondary signs and symptoms thereof, including promoting, inducing or maintaining remission of the disease.

In accordance with methods of the invention, the present inventors have unexpectedly discovered that expression of ACE2 mRNA in pancreas tissue, normally at a relatively low level in a healthy pancreas, is greatly increased in a CP disease condition. This finding is reported in greater detail in Example 1 below, and suggests that high levels of ACE2 mRNA expression are associated with CP. It is concluded that administration of an ACE2 inhibitor to counteract these high levels of ACE2 mRNA expression may provide therapeutic benefit to a CP patient.

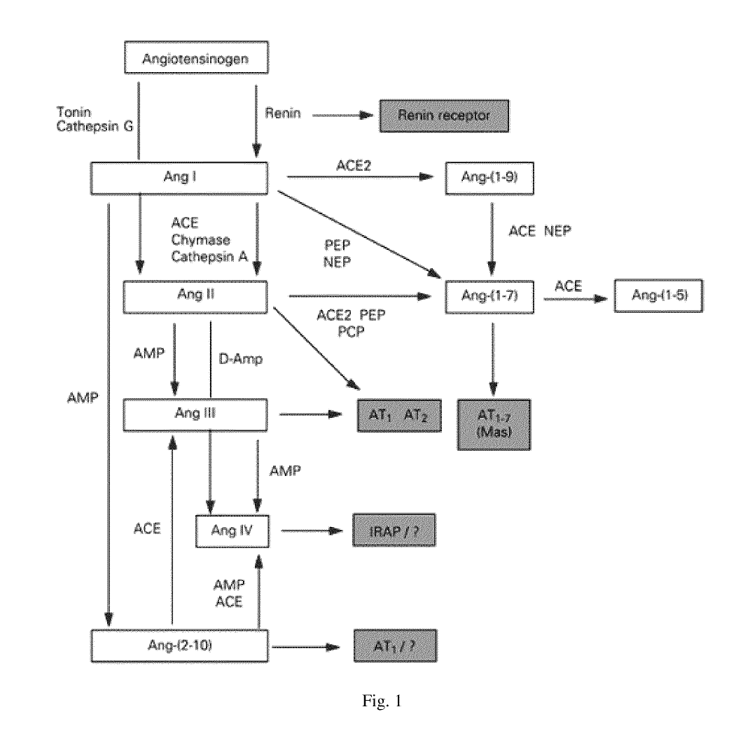

The present inventors have further found that an ACE2 inhibitor, GL1001, inhibits TNFα induced activation of NF-κB in recombinant HeLa reporter cells. This finding is reported in greater detail in Example 2 below. The effect of an ACE2 inhibitor on the renin-angiotensin system (RAS) might be predicted to involve increase in level of Ang II (see FIG. 1), which, as indicated above is implicated in a variety of pro-inflammatory effects. The present finding, contrary to such prediction, shows that activation of NF-κB, a key mediator for synthesis of pro-inflammatory cytokines, is not promoted but inhibited by the ACE2 inhibitor.

It has further surprisingly been found that the ACE2 inhibitor GL1001 inhibits in vivo basal NF-κB dependent transcription in recombinant reporter mice. This finding is reported in greater detail in Example 3 below, and appears to further support an anti-inflammatory effect of the ACE2 inhibitor that is contrary to expectation based on present understanding of the role of ACE2 in the RAS.

It has still further surprisingly been found that the ACE2 inhibitor GL1001 inhibits LPS-induced NF-κB dependent transcription in recombinant reporter mice (as described in Example 4 below) and that this inhibition is accompanied by a decrease in plasma levels of pro-inflammatory cytokines (eotaxin, IL-1α and TNFα) and an increase in plasma level of an anti-inflammatory cytokine (IL-10). This still further supports an anti-inflammatory effect of the ACE2 inhibitor that is contrary to expectation based on present understanding of the role of ACE2 in the RAS.

In this respect, it is noteworthy that pro-inflammatory activity mediated by IL-1 and TNFα cytokines as well as anti-inflammatory activity induced by increased IL-10 have been shown to play important roles in the inflammation processes that originate and sustain gastrointestinal inflammatory diseases such as gastritis, pancreatitis and inflammatory bowel disease (IBD). In the case of pancreatitis, a large body of experimental and clinical evidence suggests that IL-1, TNFα, IL-10 and some additional cytokines (e.g., IL-6, IL-8) have great relevance in the pathogenesis of local and systemic complications of acute and chronic pancreatitis and can be used as prognostic markers in its early phase. See, for example, the review articles individually cited below and incorporated herein by reference without admission that they constitute prior art to the present invention.
Behrman & Fowler (2007) Surg. Clin. N. Am. 87:1309-1324.
Al Mofleh (2008) World J. Gastroenterol. 14(5):675-684.
Bang et al. (2008), supra.

CP is characterized by permanent destruction of the pancreas on a histological level and failure of the organ on a physiological level, brought about by inflammation and fibrotic processes. During chronic inflammation in CP, the pancreas releases pro-inflammatory cytokines including TNFα, IL-1 and IL-6 that activate pancreatic stellate cells (PSCs), and this activation in turn leads to fibrosis and further deterioration. See Behrman & Fowler (2007), supra.

PSCs are usually quiescent and regulate synthesis and degradation of the extracellular matrix proteins that comprise fibrous tissue. When activated by pro-inflammatory cytokines such as TNFα, IL-1 and IL-6 or by lipopolysaccharide (LPS), PSCs produce fibrosis. Once activated, PSCs synthesize cytokines that can perpetuate PSC activation, extracellular matrix production, and fibrosis. See, for example, Apte & Wilson (2003) Pancreas 27(4):316-320.

Regarding IL-10, its potent anti-inflammatory action is thought to help in counteracting pro-inflammatory cytokine damage produced during CP. See Demols et al. (2002) Am. J. Physiol. Gastrointest. Liver Physiol. 282:G1105-G1112.

Further, clinical observations have shown increased levels of plasma IL-10 in acute pancreatitis. See Bang et al. (2008), supra. It has been suggested that IL-10 is inhibited in early stages of the inflammation processes in pancreatitis and is induced to high levels in late processes.

In in vitro cell-based studies addressing effects of alcohol exposure, given that chronic alcohol ingestion is one of the major causes of CP, it has been observed that acute alcohol treatment of monocytes augments NF-κB activation and TNFα production and inhibits IL-10 levels. See Szabo et al. (2007) Pancreatology 7:115-123, incorporated herein by reference without admission that it constitutes prior art to the present invention.

It is, then, well established that TNFα, IL-1 and IL-6 play a key role in the initial and late stages of pathogenesis of pancreatitis, and that IL-10 anti-inflammatory activity helps to alleviate inflammation in acute and chronic pancreatitis. Consequently, a method of therapeutic intervention that would inhibit production or activity of pro-inflammatory cytokines or that could produce increased levels of IL-10 could be of great benefit to a pancreatitis, for example CP, patient.

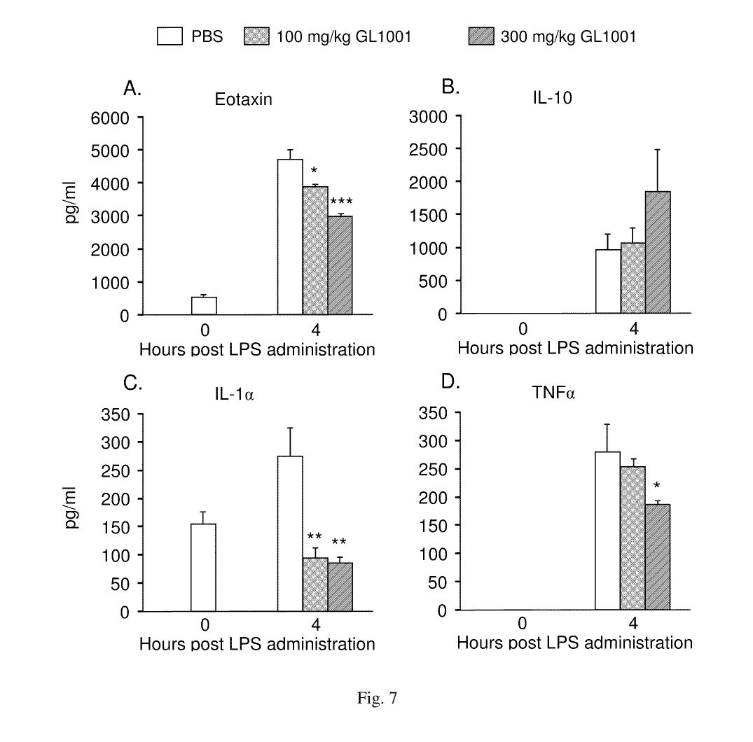

The unexpected anti-inflammatory activity of the ACE2 inhibitor GL1001 demonstrated herein is characterized by decreased systemic levels of TNFα and IL-1 cytokines with a concomitant increased systemic level of the anti-inflammatory IL-10 cytokine, in response to inflammatory conditions (see FIG. 7). Accordingly, GL1001 and other ACE2 inhibitors are believed to have potential therapeutic utility in treatment of pancreatitis, especially CP, and lends further support to conclusions drawn from the in silico investigation described in Example 1 below (see in particular Table 2).

Based in part on the above findings, the present invention provides, in one embodiment, a method for treating pancreatitis in a subject, comprising administering to the subject a therapeutically effective amount of an ACE2 inhibitor. The method is useful in both acute and chronic forms of pancreatitis, but is believed to be especially beneficial in CP.

Such therapeutically effective amount can be effective to alleviate at least one sign or symptom of pancreatitis. In one embodiment the at least one sign or symptom alleviated comprises pain, especially the upper abdominal pain that radiates toward the back that is often characteristic of CP. According to this embodiment, the ACE2 inhibitor can be administered as an alternative to or replacement for conventional analgesics such as opioids, or can supplement such conventional analgesics, for example in adjunctive or combination therapy. Alleviation of pain, besides being highly desirable in itself, can also bring further benefits such as reduction in associated conditions, for example nausea and vomiting, and psychological benefits that can improve patient compliance with respect to a therapeutic regimen, particularly one involving abstinence from alcohol consumption.

In another embodiment, particularly in the case of CP, the at least one sign or symptom alleviated comprises partial or complete loss of exocrine and/or endocrine function, or a manifestation arising from such loss. Manifestations of loss of exocrine function that can be alleviated illustratively include malabsorption of food, leading for example to excessively frequent bowel movements, greasy and/or foul-smelling feces, nutritional deficits and weight loss. Manifestations of loss of endocrine function that can be alleviated illustratively include abnormal blood sugar levels and onset of diabetes mellitus. In the present context, it will be understood that “alleviation” of loss of exocrine and/or endocrine function includes stabilization of such function, i.e., a prevention of further loss (halting loss) of function or reduction in the rate of progression (attenuation) of loss of function. Reversal of loss of exocrine and/or endocrine function can also occur, but is less likely where such loss is severe, arising from extensive destruction of pancreatic tissue by the disease.

Treatment according to the present method can be effective to slow, halt or reverse progressive fibrosis of the pancreas associated with CP. Such beneficial effects on fibrosis may be a primary effect of the treatment or may be secondary to an anti-inflammatory effect of the treatment. It is noted, however, that the present invention is not bound by any theory as to mechanism of action.

In one embodiment, a method for treating CP as described herein further comprises surgery, for example to reopen a pancreatic duct blocked by fibrotic tissue or to excise an irreversibly damaged portion of the pancreas.

Progression of CP greatly increases risk of the patient developing pancreatic cancer. Accordingly, in one embodiment, a method is provided for reducing risk of pancreatic cancer in a subject having CP, comprising administering an ACE2 inhibitor to the subject in an amount effective at least to slow progression of the CP. By reducing risk of pancreatic cancer, the present method can increase longevity of the subject, especially considering the high mortality associated with pancreatic cancer.

The ACE2 inhibitor can be selected from any chemical class or specific compound including those described above and can be administered by a route and at a dosage as provided above. Typically the ACE2 inhibitor is administered in a pharmaceutical composition comprising the ACE2 inhibitor and at least one pharmaceutically acceptable excipient. Examples of suitable excipients are provided above.

The present invention also provides a method for managing pancreatitis, especially CP, in a subject, comprising administering a therapeutically effective amount of an ACE2 inhibitor adjunctively or in co-therapy with at least one additional agent, for example an agent addressing signs, symptoms, underlying causes, contributory factors or secondary conditions associated with the pancreatitis.

The term “therapeutic combination” herein refers to a plurality of agents that, when administered to a subject together or separately, are co-active in bringing therapeutic benefit to the subject. Such administration is referred to as “combination therapy,” “co-therapy,” “adjunctive therapy” or “add-on therapy.” For example, one agent can potentiate or enhance the therapeutic effect of another, or reduce an adverse side effect of another, or one or more agents can be effectively administered at a lower dose than when used alone, or can provide greater therapeutic benefit than when used alone, or can complementarily address different aspects, symptoms or etiological factors of a disease or condition.

For example, an ACE2 inhibitor can be administered in combination or adjunctive therapy with at least one additional agent for managing pancreatitis or a condition associated therewith. Such additional agents include without limitation (a) anti-inflammatory agents other than an ACE2 inhibitor, (b) anti-pancreatitis drugs other than an ACE2 inhibitor, (c) analgesic agents, (d) protease inhibitors, (e) antibiotics, (f) pancreatic enzyme replacements, (g) antioxidants and (h) anti-alcoholism drugs.

In one embodiment, an ACE2 inhibitor is administered in combination or adjunctive therapy with an anti-inflammatory agent other than an ACE2 inhibitor. An illustrative type of anti-inflammatory agent that can be useful in such combination or adjunctive therapy is an anti-TNFα agent such as infliximab or a COX-2 inhibitor such as celecoxib.

In another embodiment, an ACE2 inhibitor is administered in combination or adjunctive therapy with an anti-pancreatitis drug other than an ACE2 inhibitor. An illustrative type of anti-pancreatitis drug that can be useful in such combination or adjunctive therapy is a PPARγ ligand such as troglitazone, a serine protease inhibitor such as camostat, an ACE inhibitor such as captopril, enalapril, lisinopril, moexipril, quinapril or the like, or an AT₁ receptor antagonist such as candesartan, eprosartan, irbesartan, losartan, olmesartan, telmisartan, valsartan or the like. The ACE2 inhibitor can be administered in combination or adjunctive therapy with more than one such anti-pancreatitis drug, for example an ACE inhibitor and an AT₁ receptor antagonist.

In yet another embodiment, an ACE2 inhibitor is administered in combination or adjunctive therapy with an analgesic agent. An illustrative type of analgesic agent is an opioid or narcotic analgesic such as codeine, hydrocodone, morphine, oxycodone or the like.

In yet another embodiment, an ACE2 inhibitor is administered in combination or adjunctive therapy with a pancreatic enzyme supplement.

In yet another embodiment, an ACE2 inhibitor is administered in combination or adjunctive therapy with an antioxidant such as N-acetylcysteine.

In yet another embodiment, an ACE2 inhibitor is administered in combination or adjunctive therapy with an anti-alcoholism drug such as acamprosate, disulfuram, nalmefene, naltrexone or the like.

Optionally, the ACE2 inhibitor can be administered in multiple co-therapy with agents from two or more of the classes listed above.

The ACE2 inhibitor and one or more additional agents administered in combination or adjunctive therapy can be formulated in one pharmaceutical preparation (single dosage form) for administration to the subject at the same time, or in two or more distinct preparations (separate dosage forms) for administration to the subject at the same or different times, e.g., sequentially. The two distinct preparations can be formulated for administration by the same route or by different routes.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in a first container, an ACE2 inhibitor and, in a second container, an additional agent such as any of those mentioned above. In another example, the ACE2 inhibitor and the additional agent are separately packaged and available for sale independently of one another, but are co-marketed or co-promoted for use according to the invention. The separate dosage forms may also be presented to a subject separately and independently, for use according to the invention.

Depending on the dosage forms, which may be identical or different, e.g., fast release dosage forms, controlled release dosage forms or depot forms, the ACE2 inhibitor and the additional agent may be administered on the same or on different schedules, for example on a daily, weekly or monthly basis.

In one embodiment, the invention provides a therapeutic combination comprising an ACE2 inhibitor and at least one additional agent selected from (a) anti-inflammatory agents other than an ACE2 inhibitor, (b) anti-pancreatitis drugs other than an ACE2 inhibitor, (c) analgesic agents, (d) protease inhibitors, (e) antibiotics, (f) pancreatic enzyme replacements, (g) antioxidants, (h) anti-alcoholism drugs, and combinations thereof. Specific examples of such additional agents are illustratively as listed above.

The present invention also provides a method for managing alcohol-related CP in a subject, comprising administering a therapeutically effective amount of an ACE2 inhibitor to the subject in conjunction with abstinence from alcohol consumption by the subject. Such abstinence is preferably total.

### EXAMPLES

### Example 1

### ACE2 mRNA Expression in Normal and Disease States

Donoghue et al. (2000), supra, reported finding ACE2 transcripts mainly in heart, kidney and testis, out of 23 normal human tissues examined, and ACE2 protein (via immunohistochemistry) predominantly in the endothelium of coronary and intrarenal vessels and in renal tubular epithelium.

Further, Tipnis et al. (2000) J. Biol. Chem. 275(43):33238-33243 reported Northern blotting analyses showing that the ACE2 mRNA transcript is most highly expressed in testis, kidney and heart.

Komatsu et al. (2002) DNA Seq. 13:217-220 reported molecular cloning of mouse angiotensin-converting enzyme-related carboxypeptidase (mACE2) showing 83% identity with human ACE2, and Northern blot analysis showing transcripts were expressed mainly in kidney and lungs.

More recently, Gembardt et al. (2005) Peptides 26:1270-1277 analyzed ACE2 mRNA and protein expression in various normal tissues of mice and rats, reporting at least detectable levels of ACE2 mRNA in all tested organs of both species (ventricle, kidney, lung, liver, testis, gallbladder, forebrain, spleen, thymus, stomach, ileum, colon, brainstem, atrium, and adipose tissue). In both species ileum tissue showed the highest expression of ACE2 mRNA, with the mouse exceeding the rat in ACE2 mRNA expression in this organ and also in the kidney and colon.

Burrel et al. (2005) Eur. Heart J. 26:369-375 recently reported that myocardial infarction increases ACE2 expression in rat and human heart.

ACE2 mRNA expression has now been examined in various human tissues from normal and diseased subjects, using the BioExpress® System of Gene Logic Inc. This system includes mRNA expression data from about 18,000 samples, of which about 90% are from human tissues, comprising both normal and diseased samples from about 435 disease states. In brief, human tissue samples, either from surgical biopsy or post-mortem removal, were processed for mRNA expression profile analysis using Affymetrix GeneChips®. Each tissue sample was examined by a board-certified pathologist to confirm pathological diagnoses. RNA isolation, cDNA synthesis, cRNA amplification and labeling, hybridizations, and signal normalization were carried out using standard Affymetrix protocols. Computational analysis was performed using Genesis Enterprise System® Software and the Ascenta® software system (Gene Logic, Inc).

In agreement with Donoghue et al. (2000), supra, and Tipnis et al. (2000), supra, the present study showed relatively high levels of ACE2 transcripts in normal human heart, kidney and testis (data not shown). However, excluding those three normal tissues, the top 8 highest expression levels of ACE2 mRNA in 70 additional normal human tissues that were examined are listed in Table 1 below, in descending order of mean expression level (given as the “average relative level,” i.e., sample set signal level in arbitrary units, normalized to the lowest signal level in all tested samples, averaged for two different probe fragments).

TABLE 1
Relative levels of ACE2 mRNA expression in normal tissues

| Sample Set | Average Relative Level |
|---|---|
| Duodenum | 221.2 |
| Small Intestine | 167.9 |
| Gallbladder | 109.9 |
| Colon | 13.6 |
| Stomach | 10.1 |
| Ovary | 5.7 |
| Pancreas | 4.3 |
| Liver | 4.2 |

These top 8 normal tissues in Table 1 (and heart, kidney and testis as well) showed average relative levels of ACE2 mRNA expression greater than 4.0, while the remaining 62 normal tissues examined showed average relative levels less than 4.0.

As defined herein, the digestive tract includes those organs through which food or excreta pass in the course of the digestive process, but excludes those organs of the digestive system, adjacent to and connecting with the digestive tract, that store and/or secrete substances aiding in digestion, for example liver, gallbladder and pancreas. This definition is broadly consistent with that given in standard reference works such as Dorland's Illustrated Medical Dictionary, 30th ed. (2003), Saunders, Philadelphia, which defines the digestive tract as that part of the digestive system formed by the esophagus, stomach and intestines, except that for convenience herein the mouth and pharynx are also included. In a normal human adult male, the digestive tract from mouth to anus is approximately 7.5 meters long and consists of upper and lower portions with the following components:
upper digestive tract: mouth (oral or buccal cavity; includes salivary glands, oral mucosa, teeth and tongue), pharynx, esophagus (gullet) and cardia, stomach, which includes the antrum and pylorus and the pyloric sphincter;
lower digestive tract: bowel or intestines, consisting of (a) small intestine, which has three parts: duodenum, jejunum and ileum; (b) large intestine, which has three parts: cecum (including the vermiform appendix which is a diverticulum of the cecum), colon (ascending colon, transverse colon, descending colon and sigmoid flexure), and rectum; and (c) anus.

The term “gastrointestinal tract” or “GI tract” is sometimes used herein, as commonly in the art, to refer to the entire digestive tract. If used in its strict sense, meaning that part of the digestive tract formed by stomach and intestines, such use is expressly specified herein or will be required by the context.

In view of the above definition of “digestive tract” or “gastrointestinal tract,” it will be seen from Table 1 that four of the top five highest expression levels of ACE2 mRNA in normal human tissues (other than heart, kidney and testis) were in components of the gastrointestinal tract, namely (in descending order of expression level): duodenum, small intestine, colon and stomach.

Examination of ACE2 mRNA expression in disease states encompassed by the BioExpress® System showed elevation of ACE2 mRNA in only a few conditions, mainly inflammatory conditions of components of the digestive system. Thus, Table 2 shows that ACE2 mRNA expression was elevated (in descending order of average fold change vs. normal) in inflammatory conditions of the stomach (chronic gastritis), major salivary gland (excluding parotid) (chronic sialadenitis), pancreas (chronic pancreatitis) and colon (Crohn's disease, active (chronic or acute inflammation)). In contrast, the levels of ACE2 mRNA in colon with active ulcerative colitis (chronic or acute inflammation), and in small intestine with active Crohn's disease (chronic or acute inflammation), were substantially unchanged from the already significant levels in corresponding normal tissues shown in Table 1.

TABLE 2
Effects of inflammatory conditions on ACE2
mRNA expression in digestive system tissues

| | Average Fold |
|---|---|
| | Change vs. |
| Sample Set | Normal |
| Stomach, Chronic Gastritis | 8.2 |
| Major Salivary Gland (Excluding Parotid), | 7.5 |
| Chronic Sialadenitis | |
| Pancreas, Chronic Pancreatitis | 3.5 |
| Colon, Crohn's Disease, Active | 2.2 |
| (Chronic Inflammation) | |
| Colon, Crohn's Disease, Active | 1.7 |
| (Acute Inflammation) | |
| Colon, Ulcerative Colitis, Active | 0.9 |
| (Chronic Inflammation) | |
| Colon, Ulcerative Colitis, Active | 1.0 |
| (Acute Inflammation) | |
| Small Intestine, Crohn's Disease, Active | 0.4 |
| (Chronic Inflammation) | |
| Small Intestine, Crohn's Disease, Active | 0.8 |
| (Acute Inflammation) | |

The above findings taken together show that 4 of the top 11 highest expression levels of ACE2 mRNA found in normal human tissues are in components of the digestive tract, and that the majority of examined disease conditions that involve elevated ACE2 mRNA expression are inflammatory conditions of the digestive tract or of the pancreas. Accordingly, these findings suggest that high levels of ACE2 mRNA expression could be a pathogenic factor and, hence, reduction of ACE2 activity could provide therapeutic benefit, in at least some inflammatory conditions of the digestive system, particularly in the stomach (chronic gastritis), major salivary gland (chronic sialadenitis), pancreas (chronic pancreatitis) and colon (Crohn's disease with chronic or acute inflammation). Further, although ACE2 mRNA levels were not elevated in colon with ulcerative colitis or small intestine with Crohn's disease, the already substantial levels of such mRNA in normal colon and small intestine suggest at least that ACE2 activity is present and, therefore, could still constitute a pathogenic factor in these two diseased tissues.

### Example 2

### Inhibition by GL1001 of TNFα-Induced Activation of NF-κB in Recombinant HeLa Reporter Cells

In inflammatory diseases of the gastrointestinal system including IBD, gastritis and pancreatitis, inflammation is likely to depend, at least in part, on activation and nuclear translocation of NF-κB family members. See, e.g., Fichtner-Feigl et al. (2005) J. Clin. Invest. 115:3057-3071 and sources cited therein. Thus, in Th1-mediated inflammations dependent on IL-12 and/or IL-23, synthesis of these cytokines is regulated by NF-κB transcription factors. In Th2-mediated inflammations dependent on IL-4 or IL-13, synthesis of these cytokines is also dependent on NF-κB transcription factors, albeit more indirectly than that of IL-12 and IL-23. Thus one method of treating gastrointestinal inflammation can be to administer agents that inhibit NF-κB activity, and indeed Fichtner-Feigl et al. (2005), supra, have shown that NF-κB decoy oligodeoxynucleotides (ODNs) that prevent NF-κB activation of gene expression are effective in treating and preventing various models of Th1- and Th2-mediated IBD in mice, including acute trinitrobenzene sulfonic acid (TNBS) induced colitis, as assessed by clinical course and effect on Th1 cytokine production; chronic TNBS induced colitis, inhibiting both production of IL-23/IL-17 and development of fibrosis; and oxazolone induced colitis, a Th2-mediated inflammatory process.

To test the ACE2 inhibitor GL1001 for anti-inflammatory activity relevant to gastrointestinal inflammatory disease, more particularly pancreatitis, effects of the compound on activation of NF-κB dependent transcription by TNFα were examined in recombinant reporter cells containing a construct with a luciferase reporter gene under control of NF-κB dependent regulatory sequences, thereby allowing detection of NF-κB dependent transcription by measuring reporter enzyme using a conventional luciferase activity assay based on detection of generated light.

In particular, HeLa cells (American Type Culture Collection) were grown in Dulbecco's modified Eagles medium (DMEM) supplemented with 10% fetal calf serum and transiently transfected with an NF-κB-luc construct (Stratagene, Inc.), as follows (with all incubation steps at 37 C unless otherwise indicated). Cells were seeded and grown to about 70% confluency in a 10 cm cell culture dish. Plasmid DNA (10 μg) was added to 1 ml serum free DMEM media in a tube. Fugene 6 transfection reagent (30 μl) (Roche) was then pipetted slowly into the tube and the contents were gently mixed by inversion. The mixture was incubated at room temperature for 15 minutes and then added dropwise to cells in one 10 cm dish. Following incubation for 24 hours, cells were detached from the plate with Trypsin-EDTA (Gibco-BRL), transferred to wells in a clear-bottom white 96-well test plate (Fisher) with 100 μl per well serum free DMEM, at a density of 3×10⁴ cells per well, and allowed to attach overnight. Compound (GL1001) was then added to wells at a concentration of approximately 0, 0.008, 0.04, 0.2, 1.0 or 5.0 μM, followed immediately by addition of TNFα (R&D) to a final concentration of 20 ng/ml. After incubation for 6 hours, 100 μl of Bright-Glo Luciferase buffer (Promega, Cat# E2610) was added, and the plate was incubated at room temperature, with mild shaking, for 10 min. Bioluminescence was then measured using a Veritas luminometer (Turner BioSystems). Each plotted data point represents the average bioluminescence of 4 independent wells.

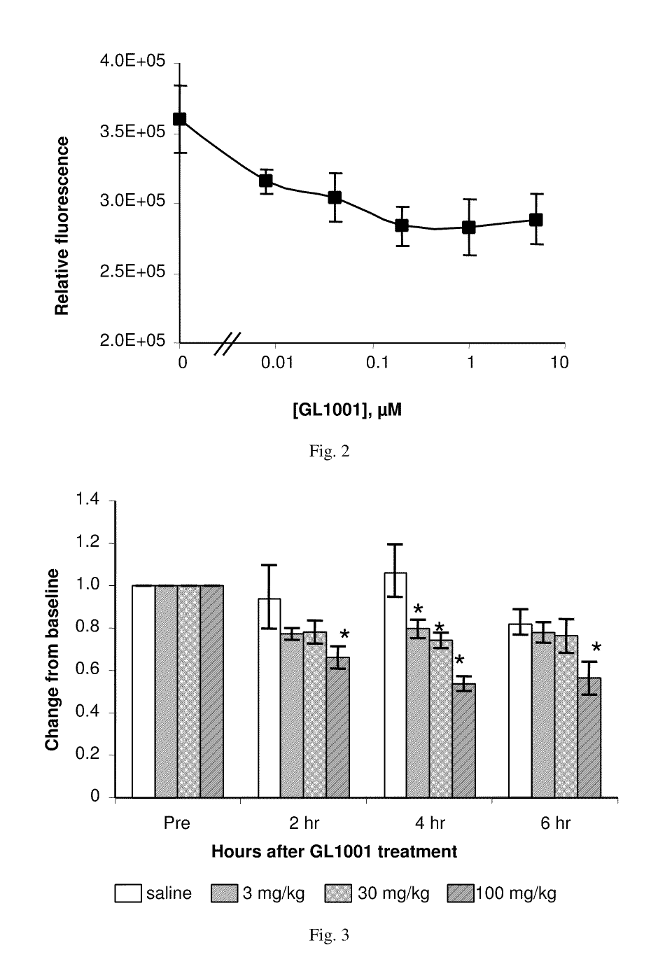

As shown in FIG. 2, GL1001 significantly inhibited TNFα induced activation of NF-κB dependent transcription at all tested concentrations, with over 80% inhibition at 8 nM and maximal inhibition over 95% at 0.2 μM. These results indicate that the ACE2 inhibitor GL1001 has potent anti-inflammatory activity, namely inhibition of activation of the NF-κB signaling pathway by the inflammatory cytokine TNFα, that is relevant to gastrointestinal inflammatory disease including pancreatitis. The present inventors are not aware of any previous report of such anti-inflammatory activity for any ACE2 inhibitor.

### Example 3

### Inhibition by GL1001 of In Vivo Basal NF-κB Dependent Transcription in Recombinant Reporter Mice

GL1001 was further tested for in vivo anti-inflammatory activity by examining its effects on basal levels of NF-κB dependent transcription in mice engineered in the germline with a construct containing an NF-κB enhancer linked to a luciferase gene (i.e., NF-κB::Luc mice), such that this NF-κB reporter construct is present in all cells of the mice.

More particularly, transgenic NF-κB::Luc mice were generated using three NF-κB response elements from the Igκ light chain promoter fused to a firefly luciferase gene as described by Carlsen et al. (2002) J. Immunol. 168:1441-1446. Pronuclear microinjection of purified construct DNA was used to generate transgenic founders in the C57BL/6 XCBA/J background. Founders were subsequently back crossed to the C57BL/6 albino background. All experimental protocols were approved by the Institutional Animal Care and Use Committee and conform to the ILAR guide for the care and used of laboratory animals. For in vivo imaging, NF-κB::Luc mice were injected intraperitoneally with luciferin (150 mg/kg) 10 minutes before imaging, anesthetized (using 1-3% isoflurane) and placed into a light-tight camera box. Mice were imaged for up to two minutes from the dorsal or ventral aspects at high-resolution settings with a field of view of 20 cm. The light emitted from the transgene was detected by an IVIS® Imaging System 200 Series (Xenogen Corporation, Alameda, Calif.), digitized and displayed on a monitor. The Living Image® software (Xenogen Corporation, Alameda, Calif.; see Rice et al. (2002) J. Biomed. Opt. 6:432-440) displays data from the camera using a pseudocolor scale with colors representing variations of signal intensity. Signal data were also quantitated and archived using the Living Image® software. Photons of light were quantitated using an oval region of interest (ROI) of varying sizes depending on the procedure, as described further below.

For luciferase assays, organs were extracted and snap frozen in liquid nitrogen. All tissue samples were placed in lysis buffer with inhibitors (Passive Lysis Buffer (Promega) and Complete Mini Protease Inhibitor Cocktail (Roche, Indianapolis, Ind.)), and were homogenized using a tissue homogenizer (Handishear, Hand-held homogenizer, VirTis, Gardiner, N.Y.). Tissue homogenates were centrifuged and clarified lysates were used for luminometer assays and western blots. For the luminometer assays, Luciferase Assay Substrate (Luciferase Assay System, Promega) was prepared as indicated by the manufacturer and placed in disposable cuvettes. Tissue homogenates (20 μl) and substrate (100 μl) were mixed and measurements were taken in a Veritas Microplate Luminometer (Turner Designs, Sunnyvale, Calif.) with the parameters of a 2 second delay, 10-second. Background luminescence readings were obtained and the background readings were subtracted from the luminescent data. Protein concentrations were determined using the BCA Protein Assay Kit (Pierce, Rockford, Ill.) following the manufacturer's protocols and analyzed using a VERSAmax tuneable microplate reader and associated Softmax Pro version 3.1.2 software (Molecular Devices, Sunnyvale Calif.). The luminescence for each of the protein lysates was calculated as arbitrary units of light per microgram of protein. Statistical analyses include MEAN, SEM and ANOVA and students t-test between treatment groups.

To test for in vivo effects of GL1001 on basal levels of NF-κB dependent transcription, male NF-κB::Luc mice were subjected to quantitative in vivo imaging of the abdominal area (using a fixed ROI of 2.76×3.7 cm) as described above, immediately before, and at 2, 4 and 6 hours after subcutaneous administration of 0, 3, 30 or 100 mg/kg GL1001 in saline. Whole-body imaging showed that GL1001 significantly inhibited basal in vivo levels of NF-κB dependent transcription of the luciferase reporter gene, primarily in the abdominal region. As shown by the quantitative imaging data in FIG. 3, at 4 hours post administration GL1001 significantly inhibited basal in vivo levels of NF-κB dependent transcription in the selected abdominal ROI by over 40% at 300 mg/kg (p<0.01 by ANOVA and Student's t-test), and to lesser but still significant extents at both lower doses.

In contrast to the results observed in NF-κB::Luc mice, no significant effect of GL1001 was observed on basal in vivo levels of reporter luciferase expression in AP-1::Luc mice constructed similarly to the present NF-κB::Luc mice (data not shown), in which reporter transcription was driven by an enhancer element responsive to activator protein-1 (AP-1), a known protooncogene thought to be involved in cell proliferation and tumor promotion.

### Example 4

### GL1001 Inhibits In Vivo LPS-Induced NF-κB Dependent Transcription in Recombinant Reporter Mice

Bacterial lipopolysaccharide (LPS), a major component of the cell wall of gram-negative bacteria, is a highly biologically active molecule which stimulates macrophages to produce and release TNFα. See, e.g., Jersmann et al. (2001) Infection and Immunity 69(3):1273-1279, and sources cited therein. One of the recognized associations of bacterial infection with gastrointestinal diseases is the activation of endothelium and upregulation of adhesion molecules. The two major proinflammatory mediators implicated in the causation of gastrointestinal diseases, bacterial LPS and TNFα have been found to cooperate to enhance the adhesive properties of endothelial cells by synergistically increasing expression of human endothelial adhesion molecules through activation of NF-κB and p38 mitogen-activated protein kinase signaling pathways.

GL1001 was further tested for in vivo anti-inflammatory activity by examining its effects on bacterial LPS induced NF-κB dependent transcription, in NF-κB::Luc mice. In particular, inflammation was induced in these mice at 6-10 weeks of age by administration of 0.5 mg/kg (i.v.) soluble LPS (sLPS; Sigma) one hour after administration of GL1001. Mice were subjected to quantitative abdominal imaging at 2, 4 and 6 h following LPS administration, as described above. In confirmatory experiments, and at the time point with the greatest modulation of luciferase signal, animals were euthanized and tissues were collected and preserved for further analysis. Luciferase signal was quantitated from several regions of interest. Statistical analyses include MEAN, SEM and ANOVA and student t-test between treatment groups.

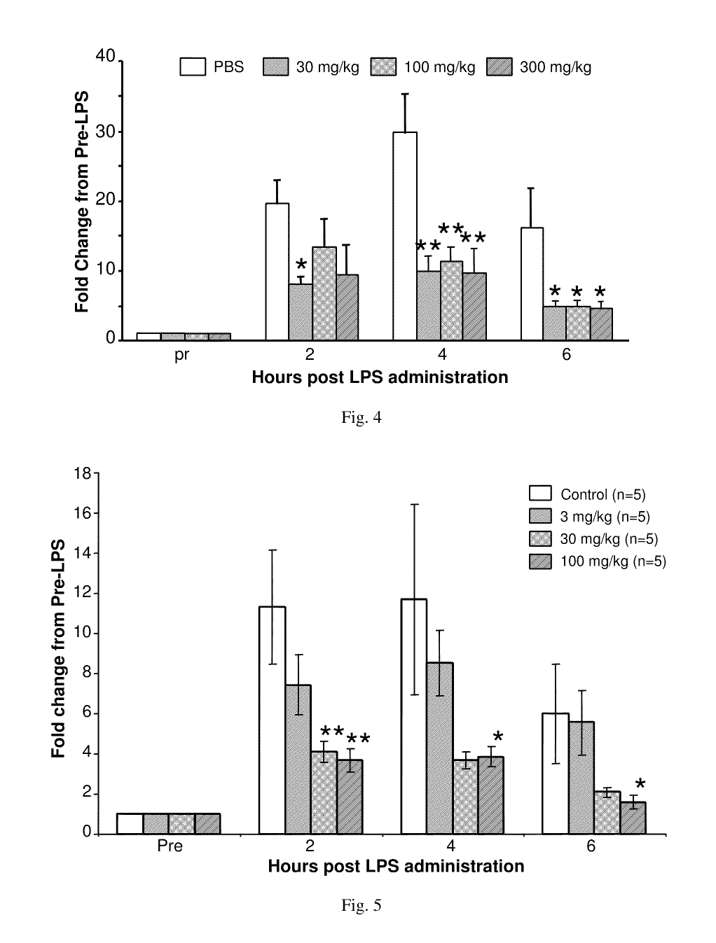

Whole-body imaging showed that GL1001 significantly inhibited LPS-induced in vivo levels of NF-κB-dependent transcription of the luciferase reporter gene, again primarily in the abdominal region. As shown by the quantitative imaging data in FIG. 4, LPS induced a strong NF-κB-dependent luciferase signal in control mice, indicating a strong NF-κB signaling response, as expected. In contrast, mice that were pretreated with GL1001 showed a significantly reduced LPS induced NF-κB signaling response, which could be measured quantitatively in the abdominal region. As inhibition of NF-κB-dependent luciferase activity was observed over the entire dose range of GL1001 in this experiment (30 mg/kg, 100 mg/kg, 300 mg/kg), the experiment was repeated using a slightly lower dose range (3-100 mg/kg). As shown in FIG. 5, in this lower dose range, GL1001 significantly reduced LPS induced NF-κB signaling at 30 and 100 mg/kg. These results show that systemic (subcutaneous) administration of the ACE2 inhibitor GL1001 showed significant in vivo anti-inflammatory activity, predominantly in the abdominal region, against bacterial LPS-induced NF-κB dependent transcription as well as against basal NF-κB dependent transcription.

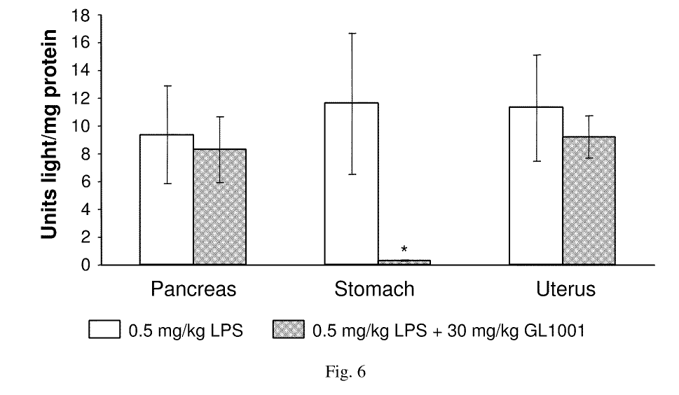

Examination of selected organs extracted from NF-κB::Luc mice treated with 0.5 mg/kg LPS and GL1001 at 30 mg/kg or with 0.5 mg/kg LPS alone (FIG. 6) showed a significant (about 37-fold) reduction of LPS induced NF-κB-dependent transcription in stomachs of GL1001 treated mice, compared to mice treated with LPS alone, but no statistically significant decrease in LPS induced NF-κB signaling in pancreas and uterus, or in any other organ or organ part that was analyzed (data not shown), namely, liver, kidney, spleen, small intestine, large intestine (colon), mesenteric lymph nodes, cecum (first part of the colon after the small intestine), ovary, uterus, submandibular lymph nodes, brain, heart and lung.

GL1001 target-mediated inhibition of LPS-induced NF-κB activity in the mouse stomach is consistent with presence of the target in stomach tissue, as evidenced by the present observation (above) of ACE2 mRNA expression in normal stomach tissue of human subjects, and by the report of ACE2 mRNA expression in the mouse stomach by Gembardt et al. (2005), supra. The fact that no inhibition of LPS induced NF-κB activity was observed in other murine tissues previously reported to express high levels of ACE2 mRNA (e.g., kidney, small intestine or colon; see Gembardt et al. (2005), supra) shows that the inhibitory effect on LPS-induced NF-κB signaling predominantly in the abdominal region following systemic (subcutaneous) administration of GL1001 is primarily due to some activity of this ACE2 inhibitor in the stomach.

### Example 5

### GL1001 Modulates Cytokines in Conjunction with Inhibition of In Vivo LPS-Induced NF-κB Dependent Transcription in Recombinant Reporter Mice

GL1001 was further tested for in vivo anti-inflammatory activity by examining its effects on various pro-inflammatory and anti-inflammatory cytokines following bacterial LPS induction of NF-κB activation in NF-κB::Luc mice. As described in Example 4, inflammation was induced in these mice (6-10 weeks of age, n=5) by administration of 1 mg/kg (i.v.) soluble LPS (sLPS; Sigma) one hour after a single subcutaneous (s.c.) administration of 100 or 300 mg/kg GL1001, or PBS (phosphate buffered saline) vehicle control. Blood samples were collected prior to experimental procedures and 4 hours post-LPS administration, and plasma was extracted and frozen for further analysis. Plasma samples were interrogated for modulation of a panel of six cytokines: eotaxin, IL-10, IL-1α, IL-4, IL-6 and TNFα, using Millipore Luminex LINCOplex mouse kits with Luminex xMap LiquiChip 200 instrumentation. Cytokine quantification was performed using standard curves and controls as suggested by the manufacturer.

Similar to the results of Example 4 seen in FIGS. 4 and 5, whole-body imaging of animals prior to and 4 hours after GL1001 administration showed that GL1001 significantly inhibited LPS-induced in vivo levels of NF-κB dependent transcription of the luciferase reporter gene, primarily in the abdomen (data not shown).

As shown in FIG. 7, plasma cytokine levels determined 4 hours post-GL1001 administration, and corresponding to the observed substantial inhibition of LPS-induced NF-κB reporter activity, showed significant and dose-dependent decrease of expression of the pro-inflammatory cytokines eotaxin, IL-1α and TNFα as compared to LPS+ vehicle treated mice. Expression of the anti-inflammatory cytokine IL-10 was enhanced at the 300 mg/kg dose of GL1001 when compared with LPS+ vehicle treated mice. IL-4 and IL-6 plasma concentrations were below Luminex detection levels and data are not presented for these in FIG. 7. For each cytokine, pre-treatment levels were combined for analysis and are shown as a single bar in FIG. 7. Statistical analysis was performed by ANOVA. Data (mean±SEM) are for cytokine concentration expressed in pg/ml as determined at the indicated times.

These results show that the initially observed suppression of NF-κB reporter activity in the abdomen of mice treated with GL1001, in an acute inflammatory model induced by bacterial LPS, is associated with a significant decrease in systemic levels of at least three pro-inflammatory cytokines and a substantial increase in the anti-inflammatory cytokine IL-10. Thus, these results strongly support the conclusion that GL1001 has significant in vivo anti-inflammatory activity and suggest that treatment with GL1001 in inflammatory conditions has a suppressive effect on inflammation signaling cascades or on adhesion pathways involving eotaxin, IL-1α, TNFα and IL-10.

### Example 6

### GL1001 Inhibits IBD Induced in Mice by Dextran Sodium Sulfate (DSS)

GL1001 was further tested for in vivo anti-inflammatory activity by examining its effects on dextran sulfate sodium (DSS) induced colitis in mice. This IBD model shows reproducible morphological changes, which are very similar to those seen in patients with ulcerative colitis. See, e.g., Hollenbach et al. (2004) FASEB J. 18(13):1550-1552. See also Bryne et al. (2006), Current Opinion in Drug Discovery & Development 8(2):207-217 and sources cited therein. These pathologies include predominant left-sided colonic inflammation, prominent regeneration of the colonic mucosa cells with dysplasia leading to colon cancer, shortening of the large intestine, focal crypt damage, and frequent lymphoid hyperplasia in both biological systems. Further, according to Hollenbach et al. (2004), supra, DSS-induced colitis in mice has a high value in assessing the efficacy of therapeutic agents commonly used in the treatment of colitis, since all therapeutically beneficial substances in human IBD were also shown to reduce the disease activity in this mouse model.

The study was designed with three groups: control (5 mice), 2.5% DSS alone (10 mice), and 2.5% DSS with GL1001 treatment (100 mg/kg subcutaneously per day) (10 mice). NF-κB::Luc mice were used to measure NF-κB activation as an indicator of inflammatory activity, as described above. In particular, organ specific luciferase activity was measured, in addition to body weight, fluid intake, occult fecal blood, organ weights and neutrophil infiltration (MPO assay). NF-κB::luc BL/6 albino background mice of 6-8 weeks of age were provided with 2.5% dextran sodium sulfate (DSS, MW 40,000; MP Biomedicals) in the drinking water. Mice were weighed, imaged and dosed with GL1001 daily. Fecal samples were collected from the bottom of the cages for each treatment group and tested for fecal consistency and occult blood using Hemocult Tape as directed by the manufacturer (Fisher Scientific) and fluid consumption was measured. At the conclusion of the study, the GI tract was removed, the various sections were cleaned and weighed, tissue samples were prepared for bioluminescent assays and myeloperoxidase (Myeloperoxidase assay kit, Cytostore) to look at neutrophil infiltration.

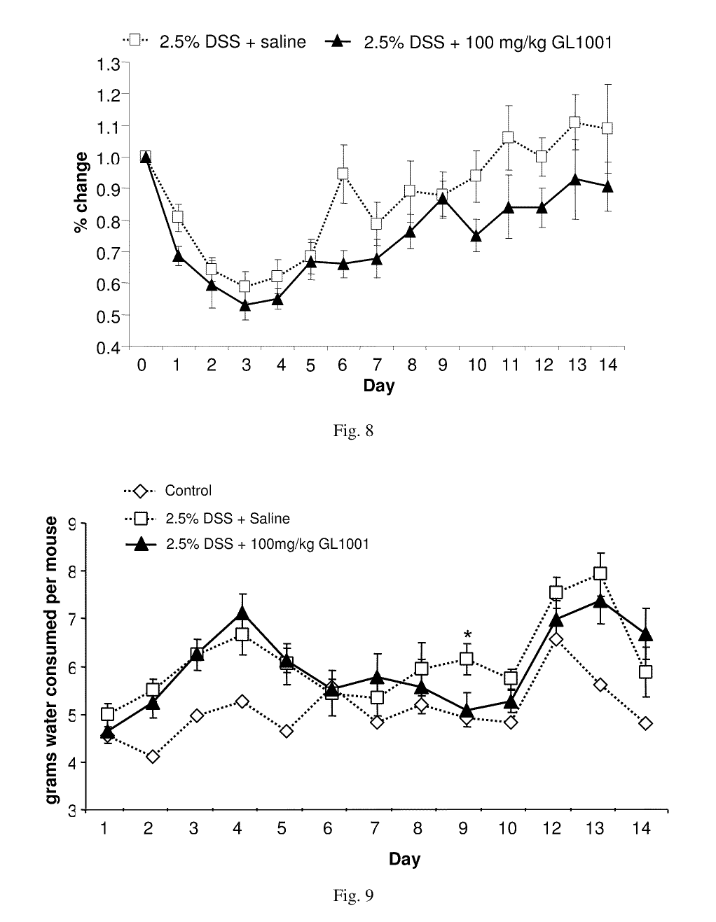

The mice were weighed and imaged at the time of daily GL1001 or vehicle control administration. Biophotonic images of the mice were acquired each day, as described above, with quantitative abdominal imaging results shown in FIG. 8. In this experiment there was an initial decrease in NF-κB-driven luciferase expression in both groups receiving DSS treatment, with a non-statistically significant divergence in luciferase expression that was maintained between the DSS only and DSS+100 mg/kg GL1001 groups throughout the experiment starting on study day 6. Water consumption was monitored for all of the animals and similar consumption rates indicate that DSS treated mice were all receiving similar amounts of DSS (FIG. 9).

IBD progression was monitored using an IBD activity index which consists of the sum of percent weight loss, stool consistency and occult fecal blood divided by 3. Table 3 shows the ranking system for each of the measured parameters.

TABLE 3
IBD activity index scoring system

| Score | Weight loss (%) | Stool consistency | Blood in feces |
|---|---|---|---|
| 0 | 0 or gain | normal | negative |
| 1 | 1-4.9 | soft | +/− |
| 2 | 5.0-9.9 | mixed (soft and | + |
| | | diarrhea) | |
| 3 | 10-15 | diarrhea | ++ |
| 4 | >15 | bloody diarrhea | gross blood |

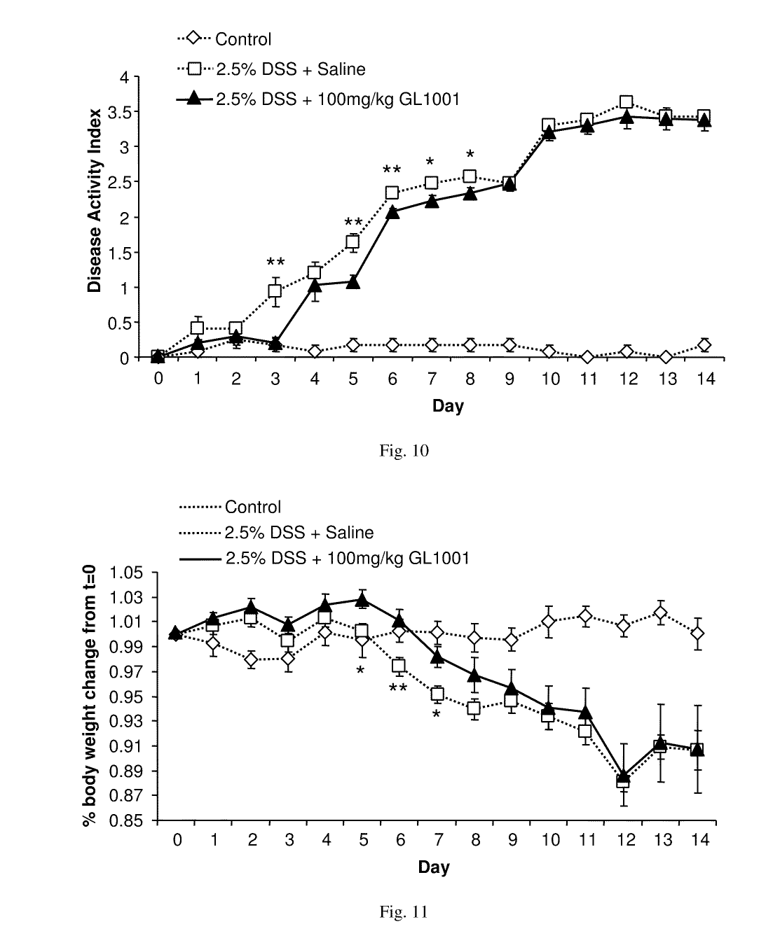

A slight delay in disease activity was seen in the GL1001 group between days 3 and 8 of the study as shown as the results for the inflammatory bowel disease activity index which are plotted in FIG. 10. The reduction in body weight was significantly delayed between days 4 and 9 in the group receiving GL1001 as compared to the DSS only treatment group (FIG. 11).

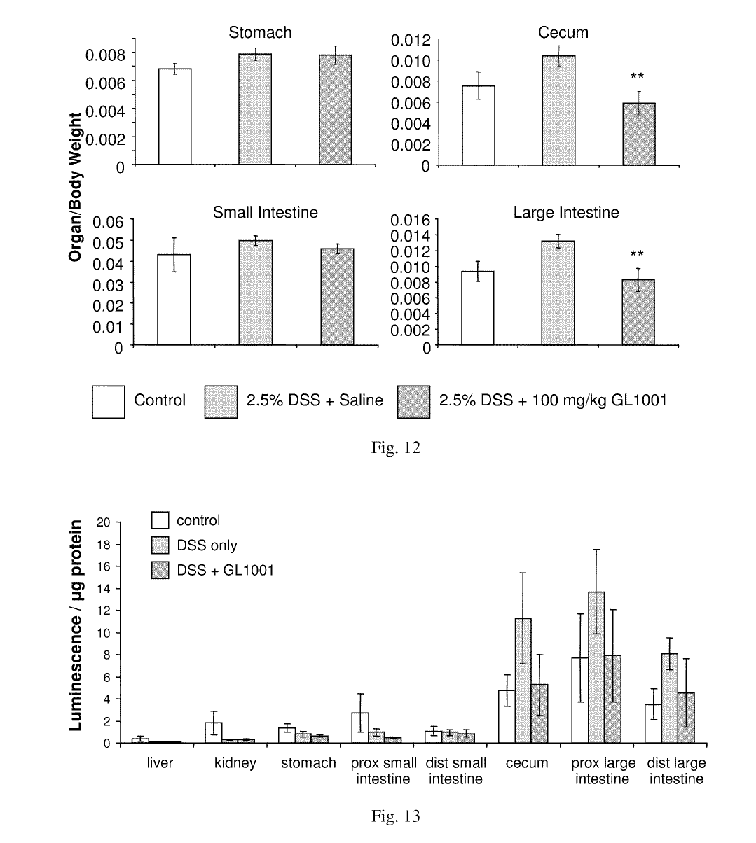

At the conclusion of the study selected organs of the gastrointestinal tract were removed, cleaned and weighed, and the ratio of organ weight to final body weight was determined. As shown in FIG. 12, significant DSS induced organ weight increases were observed, and were completely prevented by GL1001, in both the cecum and large intestine (colon), but not in the stomach or small intestine.

In addition, sections of the gastrointestinal tract, as well as liver and kidney as controls, were homogenized and luciferase expression was recorded as units of light per μg protein, in FIG. 13. Organs showing an increase in luciferase expression were the cecum and large intestine in the DSS only group. The GL1001 treated group showed luciferase expression levels similar to those in the control group that received water only.

In summary, the ACE2 inhibitor GL1001 was shown to exhibit in vivo anti-inflammatory activity in dextran sulfate sodium (DSS) induced colitis in mice, since all assays of disease-related parameters showed either significant differences or corresponding trends between the DSS and DSS+GL1001 treatment groups. The facts that systemic (subcutaneous) administration of GL1001 reduced organ weights and DSS induced NF-κB signaling in the cecum and remainder of the large intestine (colon) show that this ACE2 inhibitor has anti-inflammatory activity in portions of the gastrointestinal tract relevant to both forms of human IBD, i.e., ulcerative colitis and Crohn's disease, in addition to such activity against basal and LPS induced NF-κB signaling in the stomach.

In addition, GL1001 significantly delayed disease progression in the first week of this study, as shown by reductions in inflammatory bowel disease activity index, for instance. This activity index represents a composite assessment of three IBD symptoms, namely, weight loss, stool consistency (i.e., diarrhea), and blood in feces (i.e., bloody stools). Patients with ulcerative colitis most commonly present with bloody diarrhea, and weight loss also occurs in more severe cases. Similarly, patients with Crohn's disease generally have ongoing diarrhea and weight loss, and may also have bloody stools.

Accordingly, the present study shows that GL1001 effectively treats common symptoms of human IBD in an animal model that reportedly has high value in assessing the efficacy of therapeutic agents commonly used in the treatment of colitis, since all therapeutically beneficial substances in human IBD were also shown to reduce the disease activity in this mouse model. See, e.g., Hollenbach et al. (2004), supra.

### Example 7

### In Vivo Anti-Inflammatory Activity of GL1001 in a CP Model

As anti-inflammatory activity of GL1001 has been confirmed in an IBD model (Example 6 above), it is contemplated, in view of other experimental results reported herein, that ACE2 inhibitors such as GL1001 can be confirmed to have anti-inflammatory activity in animal models of CP. Any suitable animal model known in the art can be used, including without limitation those described in the publications individually cited below and incorporated herein by reference.

Puig-Divi et al. (1996) Pancreas 13:417-424 disclose a trinitrobenzenesulfonic acid (TBNS)-induced CP model wherein TBNS is infused into the pancreatic duct of rats, resulting in pancreatic necroinflammation followed by pancreatic fibrosis within four weeks. In TBNS-treated rats, α-SMA-positive PSCs are found in the fibrotic areas of the pancreas.

Kaku et al. (2007) Pancreas 34(3):299-309 (not admitted to be prior art to the present invention) disclose a dibutyl tin dichloride (DBTC)-induced CP model wherein a single i.v. administration of DBTC to rats results in development within one week of moderate to severe pancreatitis, followed by fibrosis within four weeks.

Van Westerloo et al. (2005) Am. J. Pathol. 166(3):721-728 disclose a cerulein-induced CP model wherein repetitive intraperitoneal injections of cerulein (50 μg/kg every hour for 6 hours, repeated three times weekly) to mice induces repeated episodes of acute pancreatitis that lead to development of pancreatic fibrosis similar to that seen in human CP.

Ohashi et al. (1990) Int. J. Pancreatol. 6(4):231-247 disclose a strain of rats (WBN/Kob) wherein characteristic lesions of CP, including acinar atrophy, inflammatory cell infiltration and fibrosis, develop spontaneously. These lesions appear focally at approximately 12 weeks of age.

Illustratively, the cerulein-induced CP model of van Westerloo et al. (2005), supra is used to determine the influence of an ACE2 inhibitor, in the present example GL1001, on pancreatic damage and fibrosis in experimental CP. Mice are given six intraperitoneal injections (hourly for 6 hours) of cerulein or saline, three times a week for 6 weeks. GL1001 is administered at a suitable dose and by a suitable route (e.g., 100 mg/kg s.c.) daily during all or a latter part (e.g., the last 3 weeks) of the 6-week cerulein injection period. Cerulein-injected mice receiving no GL1001 act as a control. The mice are sacrificed one week after the last cerulein injection. Histopathological determination of pancreatic damage is conducted on GL1001 treated and untreated mice. In particular, intrapancreatic fibrosis can be quantified by Sirius red staining, hydroxyproline content, laminate staining, number of PSCs and pancreatic level of TGFβ. Confirmation of anti-inflammatory activity of GL1001 in CP is evidenced by attenuated pancreatic damage and inflammation in mice receiving cerulein+GL1001 by comparison with mice receiving cerulein only.

All patents and publications cited herein are incorporated by reference into this application in their entirety.

The words “comprise”, “comprises”, and “comprising” are to be interpreted inclusively rather than exclusively.

1. A method for treating pancreatitis in a subject experiencing acute or chronic pancreatitis, comprising administering to the subject a therapeutically effective amount of an ACE2 inhibitor comprising a small-molecule compound or a pharmaceutically acceptable salt thereof.

2. The method of claim 1, wherein the subject has acute pancreatitis.

3. The method of claim 1, wherein the subject has chronic pancreatitis.

4. The method of claim 3, wherein the chronic pancreatitis is alcohol-induced, tropical, hereditary or idiopathic.

5. The method of claim 3, wherein the subject has a history of recurrent acute pancreatitis that developed into chronic pancreatitis.

6. The method of claim 3, wherein the ACE2 inhibitor is administered in an amount effective to alleviate pain associated with the chronic pancreatitis.

7. The method of claim 3, wherein the ACE2 inhibitor is administered in an amount effective to attenuate loss of exocrine and/or endocrine function associated with the chronic pancreatitis or to alleviate a manifestation arising from such loss.

8. The method of claim 3, wherein the ACE2 inhibitor is administered in an amount effective to slow progressive fibrosis of the pancreas.

9. The method of claim 1, wherein said therapeutically effective amount comprises a dosage amount of the ACE2 inhibitor of about 0.5 to about 5000 mg/day.

10. The method of claim 1, wherein said therapeutically effective amount comprises a dosage amount of the ACE2 inhibitor of about 5 to about 1000 mg/day.

11. The method of claim 1, wherein the ACE2 inhibitor is administered orally, buccally, sublingually, transmucosally, intranasally, intraocularly, rectally, vaginally, transdermally, parenterally, by inhalation or by implantation.

12. The method of claim 1, wherein the ACE2 inhibitor is administered in a pharmaceutical composition comprising the ACE2 inhibitor and at least one pharmaceutically acceptable excipient.

13. The method of claim 1, wherein the ACE2 inhibitor exhibits in vitro an ACE2 IC₅₀ and/or an ACE2 Kᵢ not greater than about 1000 nM.

14. The method of claim 1, wherein the ACE2 inhibitor exhibits in vitro an ACE2 IC₅₀ and/or an ACE2 Kᵢ not greater than about 100 nM.

15. The method of claim 1, wherein the ACE2 inhibitor exhibits selectivity for ACE2 versus ACE, as expressed by the ratio of IC₅₀(ACE) to IC₅₀(ACE2), of at least about 10³.

16. The method of claim 1, wherein the ACE2 inhibitor exhibits selectivity for ACE2 versus ACE, as expressed by the ratio of IC₅₀(ACE) to IC₅₀(ACE2), of at least about 10⁴.

17. (canceled)

18. (canceled)

19. (canceled)

20. The method of claim 1, wherein the compound has the formula 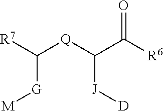
wherein
R⁶ is hydroxyl or a protecting prodrug moiety;
R⁷ is hydrogen, carboxylic acid, ether, alkoxy, an amide, a protecting prodrug moiety, hydroxyl, thiol, heterocyclyl, alkyl or amine;
Q is CH₂, O, NH or NR³, wherein R³ is substituted or unsubstituted C₁₋₅ branched or straight chain alkyl, C₂₋₅ branched or straight chain alkenyl, substituted or unsubstituted acyl, aryl or a C₃₋₈ ring;
G is a covalent bond or a CH₂, ether, thioether, amine or carbonyl linking moiety;
M is heteroaryl, substituted with at least one subanchor moiety comprising a substituted or unsubstituted cycloalkyl or aryl ring, linked thereto through a sublinking moiety (CH₂)ₙ or (CH₂)ₙO(CH₂)ₙ where n is an integer from 0 to 3;
J is a bond or a substituted or unsubstituted alkyl, alkenyl or alkynyl moiety; and
D is alkyl, alkenyl, alkynyl, aryl or heteroaryl, optionally linked to G or M to form a ring.

21. The method of claim 20, wherein, in the formula for the compound:
R⁶ is hydroxyl;
R⁷ is carboxylic acid;
Q is NH;
G is CH₂;
M is imidazolyl, thienyl, triazolyl, pyrazolyl or thiazolyl, linked through a (CH₂)ₙ or (CH₂)O(CH₂) sublinking moiety, where n is an integer from 0 to 3, to a subanchor moiety that is C₃₋₆ cycloalkyl, phenyl, methylenedioxyphenyl, naphthalenyl, or phenyl having 1 to 3 substituents independently selected from halo, C₁₋₆ alkyl, C₃₋₆ cycloalkyl, trifluoromethyl, C₁₋₆ alkoxy, trifluoromethoxy, phenyl, cyano, nitro and carboxylic acid groups;
J is a bond or CH₂ moiety; and
D is C₁₋₆ alkyl, C₃₋₆ cycloalkyl or phenyl.

22. The method of claim 20, wherein the compound is present in the (S,S)-configuration.

23. The method of claim 22, wherein the compound is substantially enantiomerically pure.

24. The method of claim 1, wherein the ACE2 inhibitor comprises a compound in the (S,S)-configuration selected from the group consisting of
2-[1-carboxy-2-[3-(4-trifluoromethylbenzyl)-3H-imidazol-4-yl]ethylamino]-4-methylpentanoic acid;
2-[1-carboxy-2-[3-naphthalen-1-ylmethyl-3H-imidazol-4-yl]ethylamino]-4-methylpentanoic acid;
2-[1-carboxy-2-[3-(4-chlorobenzyl)-3H-imidazol-4-yl]ethylamino]-4-methylpentanoic acid;
2-[1-carboxy-2-[3-(3,4-dichlorobenzyl)-3H-imidazol-4-yl]ethylamino]-4-methylpentanoic acid;
2-[1-carboxy-2-[3-(4-cyanobenzyl)-3H-imidazol-4-yl]ethylamino]-4-methylpentanoic acid;
2-[1-carboxy-2-[3-(3-chlorobenzyl)-3H-imidazol-4-yl]ethylamino]-4-methylpentanoic acid;
2-[1-carboxy-2-[3-(3,5-dichlorobenzyl)-3H-imidazol-4-yl]ethylamino]-4-methylpentanoic acid;
2-[1-carboxy-2-[3-(4-methylbenzyl)-3H-imidazol-4-yl]ethylamino]-4-methylpentanoic acid;
2-[1-carboxy-2-[3-(3,4-dimethylbenzyl)-3H-imidazol-4-yl]ethylamino]-4-methylpentanoic acid;
2-[1-carboxy-2-[3-(3-methylbenzyl)-3H-imidazol-4-yl]ethylamino]-4-methylpentanoic acid;
2-[1-carboxy-2-[3-(3,5-dimethylbenzyl)-3H-imidazol-4-yl]ethylamino]-4-methylpentanoic acid;
2-[1-carboxy-2-[3-(4-trifluoromethoxybenzyl)-3H-imidazol-4-yl]ethylamino]-4-methyl-pentanoic acid;
2-[1-carboxy-2-[3-(4-isopropylbenzyl)-3H-imidazol-4-yl]ethylamino]-4-methylpentanoic acid;
2-[1-carboxy-2-[3-(4-tert-butylbenzyl)-3H-imidazol-4-yl]ethylamino]-4-methylpentanoic acid;
2-[1-carboxy-2-[3-(4-nitrobenzyl)-3H-imidazol-4-yl]ethylamino]-4-methylpentanoic acid;
2-[1-carboxy-2-[3-(2,3-dimethoxybenzyl)-3H-imidazol-4-yl]ethylamino]-4-methylpentanoic acid;
2-[1-carboxy-2-[3-(2,3-difluorobenzyl)-3H-imidazol-4-yl]ethylamino]-4-methylpentanoic acid;
2-[1-carboxy-2-[3-(2,3-dichlorobenzyl)-3H-imidazol-4-yl]ethylamino]-4-methylpentanoic acid;
2-[1-carboxy-2-[3-(3-trifluoromethylbenzyl)-3H-imidazol-4-yl]ethylamino]-4-methylpentanoic acid;
2-[2-(3-benzo[1,3]dioxol-5-ylmethyl-3H-imidazol-4-yl)-1-carboxyethylamino]-4-methylpentanoic acid;
2-[1-carboxy-2-[3-(2-cyclohexylethyl)-3H-imidazol-4-yl]ethylamino]-4-methylpentanoic acid;
2-[1-carboxy-2-[3-phenethyl-3H-imidazol-4-yl]ethylamino]-4-methylpentanoic acid;
2-[1-carboxy-2-[3-(3-iodobenzyl)-3H-imidazol-4-yl]ethylamino]-4-methylpentanoic acid;
2-[1-carboxy-2-[3-(3-fluorobenzyl)-3H-imidazol-4-yl]ethylamino]-4-methylpentanoic acid;
2-[1-carboxy-2-[3-benzyloxymethyl-3H-imidazol-4-yl]ethylamino]-4-methylpentanoic acid;
2-[1-carboxy-2-[3-(4-butylbenzyl)-3H-imidazol-4-yl]ethylamino]-4-methylpentanoic acid;
2-[1-carboxy-2-[3-(2-methylbenzyl)-3H-imidazol-4-yl]ethylamino]-4-methylpentanoic acid;
2-[1-carboxy-2-[2-phenylthiazol-4-yl]ethylamino]-4-methylpentanoic acid;
2-[1-carboxy-2-[1-benzyl-1H-pyrazol-4-yl]ethylamino]-4-methylpentanoic acid;
2-[1-carboxy-2-[3-(2-methylbiphenyl-3-ylmethyl)-3H-imidazol-4-yl]ethylamino]-4-methylpentanoic acid;
and pharmaceutically acceptable salts thereof.

25. The method of claim 22, wherein the ACE2 inhibitor comprises the compound of formula 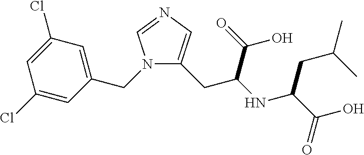
in the (S,S)-configuration, or a pharmaceutically acceptable salt thereof.

26. (canceled)

27. (canceled)

28. A method for managing pancreatitis in a subject experiencing acute or chronic pancreatitis, comprising administering a therapeutically effective amount of an ACE2 inhibitor adjunctively with at least one additional agent for managing pancreatitis.

29. The method of claim 28, wherein the ACE2 inhibitor comprises the compound of formula 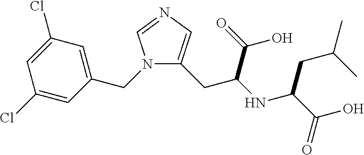
in the (S,S)-configuration, or a pharmaceutically acceptable salt thereof.

30. The method of claim 28, wherein the at least one additional agent is selected from the group consisting of anti-inflammatory agents other than an ACE2 inhibitor, anti-pancreatitis drugs other than an ACE2 inhibitor, analgesic agents, protease inhibitors, antibiotics, pancreatic enzyme replacements, antioxidants, anti-alcoholism drugs and combinations thereof.

31. The method of claim 28, wherein the ACE2 inhibitor is administered adjunctively with an anti-inflammatory agent comprising an anti-TNFα agent.

32. The method of claim 31, wherein the anti-TNFα agent comprises infliximab.

33. The method of claim 28, wherein the ACE2 inhibitor is administered adjunctively with an analgesic agent comprising an opioid.

34. The method of claim 28, wherein the ACE2 inhibitor and the at least one additional agent are separately formulated for administration at the same or different times.

35. The method of claim 28, wherein the ACE2 inhibitor and the at least one additional agent are co-formulated in a single dosage form.

36. A method for treating alcohol-related pancreatitis in a subject experiencing acute or chronic pancreatitis, comprising administering a therapeutically effective amount of an ACE2 inhibitor to the subject in conjunction with abstinence from alcohol consumption by the subject.

37. The method of claim 36, wherein the ACE2 inhibitor comprises the compound of formula 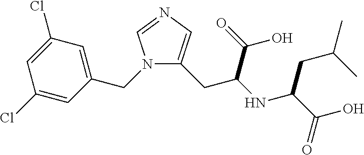
in the (S,S)-configuration, or a pharmaceutically acceptable salt thereof.
